(19) United States
(12) Patent Application Publication
(10) Pub. No.: US 2024/0115623 A1
(43) Pub. Date: Apr. 11, 2024

(54) METHOD OF EXPANDING A COMPLEX COMMUNITY OF MICROORGANISMS

(71) Applicants: MaaT PHARMA, LYON (FR); UNIVERSITE CLERMONT AUVERGNE, CLERMONT-FERRAND (FR); INISTITUT NATIONAL DE RECHERCHE POUR L'AGRICULTURE, L'ALIMENTATION ET L'ENVIORNNEMENT, PARIS (FR)

(72) Inventors: Hervé AFFAGARD, LYON (FR); Carole SCHWINTNER, LYON (FR); Cécile VERDIER, CLERMONT-FERRAND (FR); Sylvain DENIS, CLERMONT-FERRAND (FR); Jean-François BRUGERE, ISSOIRE (FR); Monique ALRIC, SAINT FLOUR L'ETANG (FR)

(21) Appl. No.: 18/258,584

(22) PCT Filed: Dec. 23, 2021
(86) PCT No.: PCT/EP2021/087619

§ 371 Date: Jun. 21, 2023

(30) Foreign Application Priority Data
Dec. 23, 2020 (EP) 20306685.7

(51) Int. Cl. A61K 35/74; A61K 35/742; A61K 35/744; A61K 35/745; A61K 35/747; C12N 1/20

(52) U.S. Cl. CPC A61K 35/74; A61K 35/742; A61K 35/744; A61K 35/745; A61K 35/747; C12N 1/20

(57) ABSTRACT

A method of expanding a complex community of microorganisms is provided, an expanded complex community of microorganisms obtained by the method is provided, a pharmaceutical formulation comprising the expanded complex community of microorganisms is provided, and the use of the expanded complex community of microorganisms and the pharmaceutical formulation is provided.

### CROSS-REFERENCE TO RELATED APPLICATIONS

This application is a National Stage of International Application No. PCT/EP2021/087619 having an International Filing Date of 23 Dec. 2021, which designated the United States of America, and which International Application was published under PCT Article 21(2) as WO Publication No. 2022/136694, which claims priority from and the benefit of European Patent Application No. 20306685.7, filed on 23 Dec. 2020, the disclosures of which are incorporated herein by reference in their entireties.

### BACKGROUND

### Field

The present disclosure concerns a method of expanding a complex community of microorganisms, an expanded complex community of microorganisms obtained by said method, a pharmaceutical formulation comprising said expanded complex community of microorganisms and the use of said expanded complex community of microorganisms and said pharmaceutical formulation.

### Brief Description of Related Developments

Complex communities of microorganisms play a key role in health and diseases. In particular, it has been discovered that the use, such as administration or transplantation, of a complex community of microorganisms may treat infections and diseases.

In case of use of a complex community of microorganisms it is important for the used sample to have an appropriate profile in terms of viability and diversity of the bacteria.

Current applied methods are often empirical and take no particular precaution to ensure the diversity of the bacteria present in the used samples, or to best preserve the viability of the bacteria.

Furthermore, depending on the use, multiple uses of complex community of microorganisms are necessary. However, the initial sample collection does not provide enough material for a full course of treatment with reproducible doses overtime.

Appealing multiple donors to obtain the desired amount of complex community of microorganisms does not itself sufficiently remedy to the prior art drawbacks. Firstly, donor selection may be fastidious and difficult, as donors have to be exempt of several well-known pathogenic microbial species. The donor selection is highly controlled, and more than 90% of potential donors do not pass the screening evaluations. Secondly, administrating different doses coming from different donors which are not homogeneous may be detrimental to the treatment efficacy. For example, in case of treatment, in order to ensure that the subject responds in the same way to each dose (or sample) of complex community of microorganisms received, it may be necessary for the doses to be as homogenous as possible.

Thus, in the existing methods, a considerable heterogeneity exists between the samples to be used which is highly undesirable.

There is thus a need to provide a method providing a larger quantity of an initial complex community of microorganisms that is safe (does not need to test many donors), reproducible (there is homogeneity between the different doses/samples obtained by following the same protocol), efficacious, easy to implement, in particular at industrial scale while the diversity of the bacteria is preserved or optimized depending on the intended purpose and the viability of the bacteria throughout the method and during storage is preserved.

The present disclosure responds to the above-described needs.

Therefore, a purpose of the present disclosure is to provide an expanded complex community of microoganisms having optimal diversity and sufficient bacterial viability for use thereof for example in administration or transplantation methods, and which can be produced safely, easily, reliably and reproducibly even at industrial scale.

### SUMMARY

The present disclosure relates to a method of expanding a complex community of microorganisms comprising the following steps:
(a) cultivating said complex community of microorganisms in at least two bioreactors, preferably in at least three bioreactors, wherein said bioreactors have at least one different parameter, said parameter being selected from pH, temperature, pressure, cultivation time, retention time, gassing conditions, redox potential, and combination thereof, to obtain at least two harvested inocula,
(b) mixing the at least two harvested inocula obtained in step (a) to obtain an expanded complex community of microorganisms.

The present disclosure also relates to an expanded complex community of microorganisms obtained by the method of the present disclosure, characterized in that it comprises at least three bacterial phyla selected from Bacteroidetes, Synergistetes, Firmicutes, Proteobacteria, Actinobacteria, Verrucomicrobia, Tenericutes, Fusobacteria and mixtures thereof.

The present disclosure also relates to an expanded complex community of microorganisms obtained by the method according to the present disclosure, characterized in that it comprises at least 15 genera selected from the group comprising Clostridium, Eubacterium, Anaerostipes, Bacteroides, Bifidobacterium, Blautia, Butyricicoccus, Collinsella, Dialister, Dorea, Eisenbergiella, Escherichia-Shigella, Faecalibacterium, Fusicatenibacter, Hungatella, Lachnoclostridium, Parasutterella, Roseburia, Eubacterium, [Eubacterium] coprostanoligenes group, [Eubacterium] fissicatena group, [Ruminococcus] torques group, Allisonella, Bilophila, Christensenella, Christensenellaceae R-7 group, Clostridium, Clostridium sensu stricto 1, Coporococcus, Desulfovibrio, Enterococcus, Flavonifractor, Lachnospiraceae NK4A136 group, Lachnospiraceae UCG-001, Lactobacillus, Parabacteroides, Peptostreptococcus, Romboutsia, Ruminoclostridium, Ruminococcaceae UCG-003, Ruminococcaceae UCG-O]3, Ruminococcaceae UCG-O]4, Ruminococcus, Subdoligranulum and Sutterella and mixtures thereof.

The present disclosure also relates to a pharmaceutical formulation comprising the expanded complex community of microorganisms obtained by the method of the present disclosure.

The present disclosure also relates to an expanded complex community of microorganisms obtained by the method of the present disclosure, the expanded complex community of microorganisms as previously defined or the pharmaceutical formulation as previously defined for its use as a medicament optionally in combination with drug-based therapy, immunotherapy, immune checkpoint inhibitors, therapy targeting tumor neoantigens, chemotherapy and/or radiation therapy.

The present disclosure also relates to an expanded complex community of microorganisms obtained by the method of the present disclosure, the expanded complex community of microorganisms as previously defined or the pharmaceutical formulation as previously defined for its use in the prevention and/or treatment of septicaemia, septic shock, infection such as Clostridioides difficile infection and associated diarrhoea (CDI), ulcerative colitis, inflammatory bowel disease (IBD), irritable bowel syndrome (IBS), idiopathic constipation, Coeliac disease, Crohn's disease, type I diabetes, type II diabetes, food allergies, solid and liquid cancers, Graft-versus-host disease (GvHD) such as gastrointestinal acute GvHD, steroid-resistant GvHD or steroid-dependant GvHD, obesity and morbid obesity, autism, sclerosis, chronic vaginal infection such as cystitis and mycosis, bone and joint infections, Parkinson's disease, Alzheimer's disease, schizophrenia and bipolar disorders, gastrointestinal disorders such as intestinal inflammation, diarrhoea, traveller's diarrhoea, mucositis, abdominal pain and gastrointestinal bleeding, intestinal dysbiosis such as intestinal dysbiosis associated with cancer chemotherapy or immunotherapy, intensive care unit (ICU) related dysbiosis, alcoholic and non-alcoholic liver disease, treatment-induced inflammation such as treatment-induced gut inflammation, inflammation induced by anti-cancer therapy, hematologic disease, viral coronavirus infections, infectious or non-infectious complications resulting from an allogenic hematopoietic stem cell transplantation (allo-HSCT) such as steroid-refractory graft versus host disease (SR-aGvHD) following allogenic hematopoietic stem cell transplantation (allo-HSCT), carrying or infection by multi-resistant bacteria including Clostridium, Enterococci such as vancomycin-resistant enterococci (VRE) and Glycopeptide-resistant Enterococci (GRE), bacteria producing extended-spectrum beta-lactamases (ESBL), carbapenemase-producing Enterobacteriaceae (CPE), Emerging Bacteria Highly Resistant to Antibiotics (BHRe), and/or malignant hemopathy, and combination thereof, optionally in combination with drug-based therapy, immunotherapy, immune checkpoint inhibitors, therapy targeting tumor neoantigens, chemotherapy and/or radiation therapy.

The present disclosure also relates to an expanded complex community of microorganisms obtained by the method of the present disclosure, the expanded complex community of microorganisms as previously defined or the pharmaceutical formulation as previously defined for its use microbiome ecosystem therapy, optionally in combination with drug-based therapy, immunotherapy, immune checkpoint inhibitors, therapy targeting tumor neoantigens, chemotherapy and/or radiation therapy.

### DETAILED DESCRIPTION

A first object of the present disclosure relates to a method of expanding a complex community of microorganisms comprising the following steps:
(a) cultivating said complex community of microorganisms in at least two bioreactors, preferably in at least three bioreactors, wherein said bioreactors have at least one different parameter, to obtain at least two harvested inocula,
(b) mixing the at least two harvested inocula obtained in step (a) to obtain an expanded complex community of microorganisms.

By “parameter” it can be understood any parameter influencing the cultivating step. Preferably, said parameter is selected from pH, temperature, pressure, cultivation time, retention time gassing conditions, redox potential, and combination thereof. More preferably, said parameter is selected from pH, temperature, cultivation time, retention time and combination thereof.

By “pH” it is herein understood the pH set point of the bioreactor. Each bioreactor may have different pH set points.

By “temperature” it is herein understood the temperature set point of the bioreactor. Each bioreactor may have different temperature set points.

By “pressure” it is herein understood the pressure set point of the bioreactor. Each bioreactor may have different pressure set points.

By “parameter X set point” it is preferably understood the value of a parameter X measured in the bioreactors, preferably with a margin of error inferior or equal to 10%, preferably inferior or equal to 5%, more preferably inferior or equal to 1%, more preferably inferior or equal to 0.1%, and even more preferably inferior or equal to 0.01%.

By “cultivation time” it is herein understood the time for cultivating said complex community of microorganisms. The complex community of microorganisms is cultivated in at least two bioreactors.

The time for cultivating said complex community of microorganisms may be different for each bioreactor.

By “retention time”, it is herein understood the time of residence of the complex community of microorganisms inside the bioreactor. It corresponds to the cultivation time when a batch culture is realized, and to the time of renewal of the entire volume of cultivation in a continuous process. The retention time may be different for each bioreactor.

By “gassing conditions” it is herein understood the gaseous conditions in the bioreactor. Each bioreactor may have different gassing conditions.

By “redox potential” it is herein understood the redox potential of the culture medium in the bioreactor. The redox potential of the culture medium may differ from one bioreactor to another.

The present disclosure also relates to a method of expanding a complex community of microorganisms comprising the following steps:
(a) cultivating said complex community of microorganisms in at least two bioreactors, preferably in at least three bioreactors, wherein said bioreactors have different pH set points, to obtain at least two harvested inocula,
(b) mixing the at least two harvested inocula obtained in step (a) to obtain an expanded complex community of microorganisms.

The present inventors have surprisingly found that the methods of the present disclosure enable to obtain an expanded complex community of microorganisms having a profile of interest either:
wherein the bacteria diversity is preserved, i.e. having a post-culture profile similar to the baseline profile (the profile before any cultivating step), or
wherein diversity is optimized in comparison to the baseline profile, depending on the intended purposes.

By “bacterial diversity” it is understood the diversity or variability of the complex community of microorganisms measured at the level of the genus or species or OTU. Bacterial diversity can be expressed with alpha-diversity parameters to describe the complex community of microorganisms such as richness (number of taxa observed in a sample), Shannon index, Simpson index and Inverse Simpson index; and with beta-diversity parameters to compare samples of complex community of microorganisms such as Bray-Curtis index and Jaccard index.

The inventors have unexpectedly found and demonstrated that by varying parameters such as pH, temperature, pressure, cultivation time, retention time, gassing conditions, redox potential, and combination thereof during the culture step and by mixing the product of at least two cultures in specific proportions, it was possible to obtain an expanded complex community of microorganisms having a post-culture profile close to that of the initial complex (herein called “baseline profile”), close to a desired profile or enriched in terms of specific species, genera or OTU (Operational Taxonomic Unit) present in comparison to the initial complex.

By “enriched in terms of species, genera or OTU” it is herein understood the increase in terms of amounts of species, genera or OTU that were present at very low abundances and not detectable in the initial complex community of microorganisms. This enrichment comes from larger proportions of species, genera or OTU that were present at very low abundances and not detectable in the initial complex community of microorganisms, and therefore become then detectable.

In particular, thanks to the method of the present disclosure, it is possible to reconstruct a profile of interest by selecting parameters such as pH, temperature, pressure, cultivation time, retention time, gassing conditions, redox potential, and combination thereof of the at least two bioreactors and the amounts of the samples coming from the at least two bioreactors that will constitute the expanded complex community of microorganisms.

The resulting expanded complex community of microorganisms has a bacterial viability at least similar and even improved in comparison to the initial sample.

As demonstrated in the example part, the method of the present disclosure may result in an unexpected increase in the diversity of the expanded complex community of microorganisms obtained by said method. The method of the present disclosure may also conduct to homogeneous expanded complex communities of microorganisms close to the baseline or close to a desired profile in terms of taxonomic profile. This homogeneity is of fundamental importance for the characterization of the final product as an industrial product such as a pharmaceutical product or a food product.

The 16S rDNA sequencing of the examples of the present disclosure demonstrates that the expanded complex community of microorganisms obtained according to the present disclosure have:
improved or preserved diversity in comparison to the initial complex community of microorganisms,
significantly good preservation of certain taxa of interest such as taxa constituting the core microbiota (Ruminococcus, Faecalibacterium, Dorea, Corprococcus, Blautia, Alistipes, Bacteroides, Subdoligranulum, Roseburia, Parabacteroides and Lachnospira), Actinobacteria, which include the well-known genus Bifidobacterium, and Firmicutes, which include the anti-inflammatory genus Faecalibacterium,
a good level of homogeneity between the obtained samples of expanded complex community of microorganisms.

By “homogeneous” or “homogeneity” it is preferably understood having a percentage of similarity superior or equal to 10%, preferably superior or equal to 20%, preferably superior or equal to 30%, preferably superior or equal to 40%, preferably superior or equal to 50%, preferably included between 50% and 100%, preferably between 75% and 95% and even more preferably between 80% and 90%.

In general, the method according to the disclosure has improved reproducibility with respect to that of the prior art, which is very important for a method intended for the manufacture of medicaments. The patient can thus receive the same product at several treatments, if more than one treatment is necessary.

As used herein, the term “at least one” refers to one or more, preferably one, two, three, four, five, six, seven, eight, nine, ten, eleven, twelve, thirteen, fourteen, fifteen, sixteen, seventeen, eighteen, nineteen or twenty, more preferably one, two, three, four, five, six, seven, eight, nine or ten, more preferably one, two, three, four or five, and even more preferably one, two, three or four.

As used herein, the term “at least two” refers to two or more, preferably two, three, four, five, six, seven, eight, nine, ten, eleven, twelve, thirteen, fourteen, fifteen, sixteen, seventeen, eighteen, nineteen or twenty, more preferably two, three, four, five, six, seven, eight, nine or ten, more preferably two, three, four or five, and even more preferably two, three, or four.

As used herein, the term “at least three” refers to three or more, preferably three, four, five, six, seven, eight, nine, ten, eleven, twelve, thirteen, fourteen, fifteen, sixteen, seventeen, eighteen, nineteen or twenty, more preferably three, four, five, six, seven, eight, nine or ten, more preferably three, four or five, and even more preferably three or four.

As used herein, the term “at least once” refers to one time or more, preferably one, two, three, four, five, six, seven, eight, nine, ten, eleven, twelve, thirteen, fourteen, fifteen, sixteen, seventeen, eighteen, nineteen or twenty times, more preferably one, two, three, four, five, six, seven, eight, nine or ten times, more preferably one, two, three, four or five times, more preferably one, two, three or four times, and even more preferably one, two or three times.

As used herein, the term “inoculum (plural inocula)” refers to the complex community of microorganisms which will be inoculated in bioreactors.

As used herein, the term “harvested inoculum (plural harvested inocula)” refers to the complex community of microorganisms which has been cultivated at least once within a bioreactor.

As used herein, the term “suspension” refers to a solution containing the complex community of microorganisms.

As used herein, the term “culture/cultivation” may refer to a fermentation process.

As used herein, the expression “a complex community of microorganisms” refers to any population of microorganisms comprising a high number of microorganisms of different species which live together and potentially in interaction. Microorganisms possibly present in such complex community include yeasts, bacteria, bacteriophage, archea, virus, fungi, algae, or any protozoa of different origins such as soil, vegetal, animal, bacterial, viral, fungal or human origin. The complex community of microorganisms of the present disclosure may comprise or may consist of microorganisms coming from one or more sources and/or from one or more donors.

The complex community of microorganisms of the present disclosure may come from:
a single source,
at least two sources,
a single donor,
at least two donors,
a single source and a single donor,
a single source and at least two donors,
at least two sources and a single donor, or
at least two sources and at least two donors,

As used herein, the term “source” refers to any environment from where the initial complex community of microorganisms comes from such as a soil, parts of a vegetal, parts of animal body or parts of human body, animal fluids or human fluids. In case of a human or an animal, the source may refer to any part of the body such as intestine.

As used herein, the term “donor” refers to a vegetal, an animal or a human, preferably a human.

The complex community of microorganisms may be a human or an animal microbiota. When the complex community of microorganisms is a human or an animal microbiota, the initial complex community of microorganisms may come from one or more sources of a human or an animal donor respectively.

Preferably, the complex community of microorganisms is a microbiota coming from one or more donors, more preferably an intestinal microbiota coming from one or more donors and even more preferably a faecal microbiota coming from at least one donor, preferably coming from at least two donors.

As used herein, the expression “expanding a complex community of microorganisms” refers to multiplying microorganisms by culture thereby providing a larger quantity of an initial complex community of microorganisms. Depending of the desired result, the expanded complex community of microorganisms obtained by the method of the present disclosure may have a post-culture profile close to that of the initial complex community of microorganisms (called “baseline”), close to a desired profile or enriched in terms of species, genera or OTU present in comparison to the initial complex.

As used herein, the term “baseline” refers to the profile before any cultivating step.

The donors may be pre-selected according to the method and criteria described in the prior art, such as for example in WO2019/171012 A1. For example, when the donors are human microbiota donors, the donors may be pre-selected according to the following screening criteria:
between 18 and 60 years of age; having a Body Mass Index (BMI) between 18 and 30;
having no personal history of serious infectious diseases, metabolic and neurological disorders, or depression;
having no recent history of medications that may deteriorate the composition of the intestinal microbiota;
absence of recent onset of symptoms associated with gastrointestinal disease, such as fever, diarrhoea, nausea, vomiting, abdominal pain, jaundice;
absence of history of serious infectious diseases, including AIDS, hepatitis etc.;
absence of recent travel to tropical countries;
absence of risky sexual behaviour;
absence of recent injury, piercing and/or tattoo(s);
absence of recent chronic fatigue;
absence of recent allergic reaction;
and/or optionally having a varied diet;
as described in WO2019/171012 A1.

The complex community of microorganisms cultivated in step (a) may be a sample, such as faeces, comprising or consisting of the complex community of microorganisms.

The samples comprising or consisting of the complex community of microorganisms collected from the donors may be controlled according to the method and qualitative criteria described in the prior art, such as for example in WO2019/171012 A1. For example, when sample comprises a human microbiota, the qualitative criteria of the sample may comprise sample consistency between 1 and 6 on the Bristol scale; absence of blood and urine in the sample; and/or absence of specific bacteria, parasites and/or virus, as described in WO2019/171012 A1.

The samples comprising or consisting of the complex community of microorganisms may be collected according to any method described the prior art, such as for example in WO2016/170285 A1, WO2017/103550 A1 and/or WO2019/171012 A1. Preferably, the samples comprising or consisting of the complex community of microorganism may be collected and then placed in anaerobic conditions. For example, as described in WO2016/170285 A1, WO2017/103550 A1 and/or WO2019/171012 A1, within 5 minutes following taking of the sample, the sample may be placed in an oxygen-tight collecting device.

The sample comprising or consisting of a complex community of microorganisms may be prepared according to the methods described in the prior art, such as for example in WO2016/170285 A1, WO2017/103550 A1 and/or WO2019/171012 A1.

For example, the sample may be prepared according to the method described in WO2016/170285 A1 comprising the steps of:
taking at least one sample from the donor subject;
within 5 minutes following taking of the sample, placing said sample in an oxygen-tight collecting device;
mixing the obtained sample with at least one saline aqueous solution comprising at least one cryoprotectant and/or at least one bulking agent to obtain a mixture;
optionally filtering the mixture in particular using a filter comprising pores of diameter less than or equal to 0.7 mm, preferably less than or equal to 0.5 mm; and
storing the mixture previously obtained by freezing at a temperature between −15° C. and −100° C., preferably between −60° C. and −90° C.;
all these steps may be carried out under anaerobiosis.

For example, the sample collected comprising or consisting of a complex community of microorganisms may be lyophilised according to the method described in WO2017/103550 A1 comprising the step of:
mixing the sample from a donor subject with a diluent chosen from polyols, di- to pentasaccharides, maltodextrins and mixtures thereof; and
freezing the mixture obtained at a temperature less than −50° C., preferably included between −70° C. and −100° C., then lyophilizing it.

For example, a homogeneous mixture of complex community of microorganisms coming from at least two preselected donors may be prepared according to the process described in WO2019/171012 A1 comprising the steps of:
taking at least one sample of faecal microbiota from said preselected donors;
within less than five minutes of collection, placing the obtained sample in an oxygen-tight collection device;
performing a quality control of the samples taken and exclusion of samples not meeting the quality criteria; —adding to each of the samples retained after the control step an aqueous saline solution comprising at least one cryoprotectant and/or bulking agent;
filtering the obtained samples to form a series of inocula;
grouping said inocula to form an inocula mixture; and
homogenizing said obtained mixture, in particular by manual stirring or using a stirring device; the steps may be carried out anaerobically.

As used herein, the term “a bioreactor” refers to any device including vessels useful for cultivating microorganisms in which culture parameters can be controlled (such as temperature, pH, retention time, aeration, supply etc.). In the present disclosure, the terms “fermenter” and “bioreactor” have the same meaning. The cultivating step may thus be considered as a fermentation step. The bioreactor may integrate the main parameters of different environments and thus reconstruct the environment from which the complex community of microorganisms has been collected. For example, when the complex community of microorganisms comes from an in vivo human colonic environment, the bioreactor may integrate in vivo human colonic environment parameters, such as pH, temperature, supply of ileal effluents, retention time and anaerobiosis, as described in the prior art such as for example in Cordonnier et al., 2015 (Dynamic In Vitro Models of the Human Gastrointestinal Tract as Relevant Tools to Assess the Survival of Probiotic Strains and Their Interactions with Gut Microbiota, Microorganisms. 2015 December; 3(4): 725-745).

The cultivating step may be performed in Batch, Fed-batch or using continuous culture technique as described in the prior art such as for example in Wiese, M., et al., PeerJ., 2018. Jan 19; 6: p. e4268; or Takagi, R., et al.; 35 PloS One, 2016. 11(8): p. e0160533.

The culture medium used in the bioreactor may be any culture medium as described in the prior art, such as for example in Cordonnier et al., 2015. (Dynamic In Vitro Models of the Human Gastrointestinal Tract as Relevant Tools to Assess the Survival of Probiotic Strains and Their Interactions with Gut Microbiota, Microorganisms. 2015 December; 3(4): 725-745). Preferably, each component of the culture medium meets the food regulatory standards and/or pharmaceutical regulatory standards if the expanded community is for human consumption. Preferably, the culture medium is as close as possible to the one of the environment from which the complex community of microorganisms has been collected.

The complex community of microorganisms can be cultivated in aerobic or anaerobic conditions in the bioreactor depending on the microorganisms. In case of intestinal microbiota, the complex community of microorganisms is preferably cultivated in anaerobic conditions.

An antifoam agent may be added to the culture medium. Examples of antifoam agent comprise, but are not limited to, silicon polymer, silicon emulsion, polyether dispersions, and mixtures thereof.

The complex community of microorganisms according to the present disclosure may be cultivated at a temperature included between 15° C. and 50° C., preferably between 33° C. and 40° C., more preferably between 36° C. and 38° C., depending the one of the environment from which the complex community of microorganisms has been collected.

In the present disclosure, any combination of parameters may be used, with the proviso that at least two bioreactors, preferably at least three bioreactors, having at least one different parameter are used.

In the present disclosure, when the at least one different parameter is retention time, any combination of retention time can be used with the proviso that at least two bioreactors, preferably at least three bioreactors, having different retention time are used.

Preferably, in the method of the present disclosure, said bioreactors have different retention time included between 6 hours and 102 hours, preferably between 12 hours and 96 hours, preferably between 24 hours and 72 hours. The retention time may be selected from 12 hours, 24 hours, 48 hours, 72 hours, 96 hours and combination thereof, preferably from 24 hours, 48 hours, 72 hours, and combination thereof.

Preferably, in the method of the present disclosure, at least one bioreactor has a retention time included between 6 hours and 18 hours, preferably between 9 hours and 15 hours and more preferably between 11 hours and 13 hours.

Preferably, in the method of the present disclosure, at least one bioreactor has a retention time included between 18 hours and 30 hours, preferably between 21 hours and 27 hours and more preferably between 23 hours and 25 hours.

Preferably, in the method of the present disclosure, at least one bioreactor has a retention time included between 42 hours and 54 hours, preferably between 45 hours and 51 hours and more preferably between 47 hours and 49 hours.

Preferably, in the method of the present disclosure, at least one bioreactor has a retention time included between 66 hours and 78 hours, preferably between 69 hours and 75 hours and more preferably between 71 hours and 73 hours.

Preferably, in the method of the present disclosure, at least one bioreactor has a retention time included between 90 hours and 102 hours, preferably between 93 hours and 99 hours and more preferably between 95 hours and 97 hours.

Preferably, in the method of the present disclosure:
a first bioreactor has a retention time included between 18 hours and 30 hours, preferably between 21 hours and 27 hours and more preferably between 23 hours and 25 hours, and
a second bioreactor has a retention time included between 6 hours and 18 hours, preferably between 9 hours and 15 hours and more preferably between 11 hours and 13 hours, or a retention time included between 42 hours and 54 hours, preferably between 45 hours and 51 hours and more preferably between 47 hours and 49 hours.

Preferably, in the method of the present disclosure:
a first bioreactor has a retention time included between 6 hours and 18 hours, preferably between 9 hours and 15 hours and more preferably between 11 hours and 13 hours,
a second bioreactor has a retention time included between 18 hours and 30 hours, preferably between 21 hours and 27 hours and more preferably between 23 hours and 25 hours, and
a third bioreactor has a retention time included between 42 hours and 54 hours, preferably between 45 hours and 51 hours and more preferably between 47 hours and 49 hours.

Preferably, in the method of the present disclosure:
a first bioreactor has a retention time included between 18 hours and 30 hours, preferably between 21 hours and 27 hours and more preferably between 23 hours and 25 hours, and
a second bioreactor has a retention time included between 6 hours and 18 hours, preferably between 9 hours and 15 hours and more preferably between 11 hours and 13 hours, or a retention time included between 42 hours and 54 hours, preferably between 45 hours and 51 hours and more preferably between 47 hours and 49 hours, or a retention time included between 66 hours and 78 hours, preferably between 69 hours and 75 hours and more preferably between 71 hours and 73 hours, or a retention time included between 90 hours and 102 hours, preferably between 93 hours and 99 hours and more preferably between 95 hours and 97 hours.

Preferably, in the method of the present disclosure:
a first bioreactor has a retention time included between 6 hours and 18 hours, preferably between 9 hours and 15 hours and more preferably between 11 hours and 13 hours,
a second bioreactor has a retention time included between 18 hours and 30 hours, preferably between 21 hours and 27 hours and more preferably between 23 hours and 25 hours, and
a third bioreactor has a retention time included between 42 hours and 54 hours, preferably between 45 hours and 51 hours and more preferably between 47 hours and 49 hours,
a fourth bioreactor has a retention time included between 66 hours and 78 hours, preferably between 69 hours and 75 hours and more preferably between 71 hours and 73 hours, or a retention time included between 90 hours and 102 hours, preferably between 93 hours and 99 hours and more preferably between 95 hours and 97 hours.

In the present disclosure, when the at least one different parameter is cultivation time, any combination of cultivation time can be used with the proviso that at least two bioreactors, preferably at least three bioreactors, having different cultivation time are used.

Preferably, in the method of the present disclosure, said bioreactors have different cultivation time included between 12 hours and 90 days, preferably between 24 hours and 60 days, more preferably between 48 hours and 30 days, more preferably between 72 hours and 15 days, and even more preferably between 96 hours and 7 days. The cultivation time may be selected from 12 hours, 24 hours, 48 hours, 72 hours, 96 hours, 7 days, 15 days, 30 days, 60 days and 90 days.

In the present disclosure, when the at least one different parameter is temperature, any combination of temperature can be used with the proviso that at least two bioreactors, preferably at least three bioreactors, having different temperature set points are used.

Preferably, in the method of the present disclosure, said bioreactors have different temperature set points included between 15° C. and 50° C., preferably between 33° C. and 40° C., more preferably between 36° C. and 38° C.

In the present disclosure, when the at least one different parameter is pH, any combination of pH set points may be used with the proviso that at least two bioreactors, preferably at least three bioreactors, having different pH set points are used.

Preferably, in the method of the present disclosure, said bioreactors have a different pH set points included between 4.5 and 8.0, preferably between 5 and 7.6 and more preferably between 5.2 and 7.4.

Preferably, in the method of the present disclosure, at least one bioreactor has a pH set point included between 4.5 and 5.8, preferably between 5 and 5.6 and more preferably between 5.2 and 5.4.

Preferably, in the method of the present disclosure, at least one bioreactor has a pH set point included between 5.9 and 6.8, preferably between 6 and 6.6 and more preferably between 6.2 and 6.4.

Preferably, in the method of the present disclosure, at least one bioreactor has a pH set point included between 6.9 and 8, preferably between 7 and 7.6 and more preferably between 7.2 and 7.4.

Preferably, in the method of the present disclosure:
a first bioreactor has a pH set point included between 4.5 and 5.8, preferably between 5 and 5.6 and more preferably between 5.2 and 5.4,
a second bioreactor has a pH set point included between 5.9 and 6.8, preferably between 6 and 6.6 and more preferably between 6.2 and 6.4, or a pH set point included between 6.9 and 8, preferably between 7 and 7.6 and more preferably between 7.2 and 7.4.

Preferably, in the method of the present disclosure:
a first bioreactor has a pH set point included between 5.9 and 6.8, preferably between 6 and 6.6 and more preferably between 6.2 and 6.4,
a second bioreactor has a pH set point included between 4.5 and 5.8, preferably between 5 and 5.6 and more preferably between 5.2 and 5.4, or a pH set point included between 6.9 and 8, preferably between 7 and 7.6 and more preferably between 7.2 and 7.4.

Preferably, in the method of the present disclosure:
a first bioreactor has a pH set point included between 6.9 and 8, preferably between 7 and 7.6 and more preferably between 7.2 and 7.4,
a second bioreactor has a pH set point included between 4.5 and 5.8, preferably between 5 and 5.6 and more preferably between 5.2 and 5.4, or a pH set point included between 5.9 and 6.8, preferably between 6 and 6.6 and more preferably between 6.2 and 6.4.

Preferably, in the method of the present disclosure:
a first bioreactor has a pH set point included between 4.5 and 5.8, preferably between 5 and 5.6 and more preferably between 5.2 and 5.4,
a second bioreactor has a pH set point included between 5.9 and 6.8, preferably between 6 and 6.6 and more preferably between 6.2 and 6.4, and
a third bioreactor has a pH set point included between 6.9 and 8, preferably between 7 and 7.6 and more preferably between 7.2 and 7.4.

Preferably, said bioreactors have only one or only two different parameters selected from pH and/or retention time as previously defined, the other parameters being identical between the bioreactors.

Depending on the intended result, the skilled person would be able to choose the specific combination of parameters as previously defined for each bioreactor.

Furthermore, additional compounds, such as fibers and prebiotics, can be added in the at least two bioreactors in step (a). Their concentration and nature may vary from one reactor to another depending on the intended result.

Each bioreactor may be inoculated with a sample such as faeces comprising or consisting of a complex community of microorganisms at a concentration ranging from 0.01 to 100 g of sample/L, preferably 0.05 to 15 g of sample/L, preferably at a concentration ranging from 0.1 to 10 g of sample/L, preferably at a concentration ranging from 0.1 to 5 g of sample/L, more preferably at a concentration ranging from 0.1 to 1 g of sample/L in the bioreactor.

Each bioreactor may be inoculated with a sample, such as faeces, comprising or consisting of a complex community of microorganisms at a concentration ranging from 10³ to 10¹³ bacteria/L, preferably from 10⁶ to 10¹² bacterial/L, more preferably from 10⁹ to 10¹¹ bacterial/L in the bioreactor. The method of the present disclosure may comprise additional steps such as for example a filtration step, a homogenization step, a freezing step, a thawing step and/or a lyophilisation step according to any method described in the prior art such as for example as described in WO 2016/170285 A1, WO2017/103550 A1 or WO2019/171012 A1.

These steps may be performed before step a), after step a) and before step b), after step b) and/or before and/or after any repetition of these steps.

If the complex community of microorganisms used in step (a) comes from two or more sources and/or from two or more donors, the method of the present disclosure may additionally comprise a pooling step.

Thus, at least one part or all of the complex community of microorganisms used in step (a) may be freshly collected, or may have been frozen, thawed and/or lyophilized before step (a). In the same manner, at least one or all the harvested inocula obtained in step (a) and at least one part or all of the expanded complex community of microorganisms obtained in step (b) may have been frozen, thawed and/or lyophilized.

By “freshly collected” it is herein understood a sample that has not been frozen, thawed and/or lyophilized.

At least one part or all the complex community of microorganisms used in step (a) may have been extracted, partially extracted, partially isolated, isolated, partially separated or separated from its initial matrix or not by techniques well-known for the skilled person.

At least one part or all the complex community of microorganisms used in step (a) may have been artificially synthesised or genetically modified.

The fraction of the isolated complex community of microorganisms may comprise a spore-forming bacteria. The fraction of the isolated complex community of microorganisms may be in spore form.

The term “isolated” encompasses a complex community of microorganisms that has been (1) separated from at least some of the components with which it was associated when initially produced (whether in nature or in an experimental setting), and/or (2) produced, prepared, purified, and/or manufactured.

Isolated complex community of microorganisms may be separated from at least about 10%, about 20%, about 30%, about 40%, about 50%, about 60%, about 70%, about 80%, about 90%, or more of the other components with which it was initially associated.

In some aspects, isolated complex community of microorganisms is more than about 80%, about 85%, about 90%, about 91%, about 92%, about 93%, about 94%, about 95%, about 96%, about 97%, about 98%, about 99%, or more than about 99% pure.

The term “pure” means that the complex community of microorganisms is substantially free of other components.

The terms “purify,” “purifying” and “purified” refer to a complex community of microorganisms that has been separated from at least some of the components with which it was associated either when initially produced or generated (e.g., whether in nature or in an experimental setting), or during any time after its initial production. A complex community of microorganisms may be considered purified if it is isolated at or after production, such as from a material or environment containing the complex community of microorganisms, and a purified complex community of microorganisms may contain other materials up to about 10%, about 20%, about 30%, about 40%, about 50%, about 60%, about 70%, about 80%, about 90%, or above about 90% and still be considered “isolated.”

In some aspects, the complex community of microorganisms is more than about 80%, about 85%, about 90%, about 91%, about 92%, about 93%, about 94%, about 95%, about 96%, about 97%, about 98%, about 99%, or more than about 99% pure. The complex community of microorganisms can be independently purified from one or more other complex community of microorganisms produced and/or present in the material or environment containing the complex community of microorganisms. The complex community of microorganisms may be purified from residual habitat products.

For example, the freezing may be carried out as described in the prior art and for example in WO 2016/170285 A1 or WO2017/103550 A1. In particular, the freezing may be carried out by placing the sample to freeze at a temperature between −15° C. and −100° C., preferably between −35° C. and −95° C., and more preferably between −60° C. and −90° C., optionally with at least one aqueous saline solution comprising at least one cryoprotectant and/or bulking agent, as described in WO 2016/170285 A1 or WO2017/103550 A1.

For example, the thawing may be carried out according to the methods described in the prior art and for example on page 11 of WO 2016/170285 A1 up to ambient temperature. In particular, the thawing may be carried out:
by placing the frozen sample in a water bath at a temperature included between 35° C. and 40° C., for a period of a few minutes until few hours, preferably from 1 to 20 minutes, and more preferably from 2 to 10 minutes;
by placing the frozen sample at a temperature included between 2° C. and 10° C., for example between 4° C. and 8° C., for a period from 1 to 30 hours, preferably from 10 to 20 hours; or
by placing the sample at ambient temperature for 1 to 10 hours, preferably for 2 to 4 hours.

As for the other steps, the thawing may be carried out under anaerobiosis.

A cryoprotectant is a substance used to protect the sample from damage caused by freezing, in particular due to the formation of ice crystals. The cryoprotectant may be any cryoprotectant described in the prior art and for example on pages 8 to 9 of WO2016/170285 A1 or on pages 8 to 10 of WO2017/103550 A1. Preferably, the cryoprotectant is selected from polyols, di- to penta-saccharides, Dimethyl Sulfoxide (DMSO), and mixtures thereof. More preferably, the cryoprotectant may be chosen from glycerol, mannitol, sorbitol, DMSO, propylene glycol, ethylene glycol such as polyethylene glycol, trehalose and its analogs, saccharose, galactose-lactose and mixtures thereof.

The bulking agent may be any bulking agent described in the prior art and for example on page 9 of WO2016/170285 A1 or on pages 8 to 10 of WO2017/103550 A1. Preferably, the bulking agent is a mixture of maltodextrins.

The total amount of cryoprotectant may be included between 3 and 30% by weight/volume, preferably between 4% and 20% by weight/volume and more preferably between 5% and 15% by weight/volume with respect to the total volume of the sample (which may be a suspension) comprising or consisting of the complex community of microorganisms; and/or the amount of bulking agent may be included between 3 and 30% by weight, preferably between 4 and 20% by weight and more preferably between 5% and 15% by weight with respect to the total volume of the sample (which may be a suspension) comprising or consisting of the complex community of microorganisms.

The aqueous saline solution of the present disclosure may comprise water and physiologically acceptable salts as described in the prior art and for example on pages 7 to 8 of WO2016/170285 A1 or on page 8 of WO2017/103550 A1. For example, the salts may be salts of calcium, sodium, potassium or magnesium, with chloride, gluconate, acetate or hydrogen carbonate ions and mixtures thereof, and preferably sodium chloride, calcium chloride, magnesium chloride, potassium chloride, sodium gluconate, sodium acetate and mixtures thereof.

Salts may be present in the aqueous saline solution at a concentration included between 2 and 20 g/L, preferably between 5 and 15 g/L, more preferably between 7 and 10 g/L and even more preferably 9 g/L.

The aqueous saline solution may also optionally comprise at least one antioxidant. Examples of antioxidants, comprise but are not limited to, antioxidants described on pages 7 to 8 of WO2016/170285 A1 or on pages 8 to 9 of WO2017/103550 A. For example, the antioxidant may be chosen from ascorbic acid and its salts (ascorbate), tocopherols (acid α-tocopherol), cysteine and its salt forms (such as hydrochloride) and mixtures thereof, and preferably chosen from sodium ascorbate, tocopherols, cysteine hydrochloride monohydrate, and mixtures thereof.

The antioxidant may be present in an amount included between 0.1 and 1% by weight/volume, preferably between 0.3% and 0.8% by weight/volume, and preferably between 0.4% and 0.6% by weight/volume with respect to the total volume of the sample comprising or consisting of the complex community of microorganisms.

Preferably, the aqueous saline solution comprises:
at least one salt selected from sodium chloride, calcium chloride, magnesium chloride, potassium chloride, sodium gluconate, sodium acetate, and mixtures thereof, and
optionally at least one antioxidant, preferably selected from sodium ascorbate, tocopherols, cysteine hydrochloride monohydrate, and mixtures thereof.

Typically, the aqueous saline solution is added to the sample comprising or consisting of the complex community of microorganisms with a ratio weight (g)/volume (mL) included between 1:0.5 and 1:10, preferably 1:2 and 1:8, and more preferably 1:4. A ratio weight/volume of the sample:solution equal to 0.5 weight: 10 volume means that the sample is mixed to 0.5 weight (for example 0,5 g) for 10 volume of solution (for example 10 mL).

The sample comprising or consisting of the complex community of microorganisms used in step (a) may be under the form of a suspension comprising an aqueous saline solution as previously defined.

Preferably, the sample comprising or consisting of the complex community of microorganisms used in step (a) is under the form of a suspension comprising an aqueous saline solution, at least one cyroprotectant and/or at least one bulking agent as previously defined.

Step (a) may be repeated at least once. In this case, successive cultivating steps may be carried out without mixing the harvested inocula between the successive cultivating steps. That is, the harvested inocula of each bioreactor obtained in step (a) may be re-cultivated with the same parameter such as pH, temperature, pressure, cultivation time, retention time, gassing conditions, redox potential, and combination thereof, preferably pH and/or retention time as previously defined, or with different parameters such as pH, temperature, pressure, cultivation time, retention time, gassing conditions, redox potential, and combination thereof, preferably pH and/or retention time as previously defined, as those previously used depending on the intended result. The bioreactor may be the same or different from the bioreactor previously used.

Preferably, step (a) is repeated at least once, each harvested inoculum being re-cultivated in a bioreactor, with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result.

As previously mentioned, at least one or all the harvested inocula obtained in step (a) may be frozen, optionally thawed and/or lyophilized between each subsequent step, whether or not step (a) is repeated. That is, each harvested inoculum obtained in step (a) may be frozen, optionally thawed and/or lyophilized before some or all repetition of step (a) and/or before step (b).

Preferably, at least one part or all of the complex community of microorganisms used in step (a) has been frozen at a temperature between −15° C. and −100° C., preferably between −35° C. and −95° C., and more preferably between −60° C. and −90° C., optionally with at least one aqueous saline solution comprising at least one cryoprotectant and/or bulking agent, and optionally thawed before being inoculated in step (a).

Preferably, at least one or all the harvested inocula obtained in step (a) have been frozen at a temperature at a temperature between −15° C. and −100° C., preferably between −35° C. and −95° C., and more preferably between −60° C. and −90° C., optionally with at least one aqueous saline solution comprising at least one cryoprotectant and/or bulking agent, to obtain at least one frozen harvested inoculum.

Preferably, the method of the present disclosure further comprises, before step (b), a step of thawing the at least one frozen harvested inoculum. Said thawing step may be followed by at least one step of re-cultivating the harvested inocula, with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result.

Preferably, the method of the present disclosure further comprises a step of freezing at least one part or all the expanded complex community of microorganisms obtained in step (b) at a temperature between −15° C. and −100° C., preferably between −35° C. and −95° C., and more preferably between −60° C. and −90° C., optionally with at least one aqueous saline solution comprising at least one cryoprotectant and/or bulking agent, to obtain a frozen inoculum mixture.

Steps (a) or steps (a) and (b) of the present disclosure may be repeated at least once, with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result.

Preferably, the frozen inoculum mixture is thawed followed by at least one step of re-cultivating the mixture according to step (a), with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result, and optionally by at least one step of mixing according to step (b). That is, the frozen inoculum mixture may be re-cultivated in at least two bioreactors, preferably in at least three bioreactors with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result, and the resulting harvested inocula may be optionally mixed.

The thawing may be carried out according to the method as previously described.

Thus, in the method according to the present disclosure:
steps (a) and (b) may be not repeated,
step (a) may be repeated at least once, with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result, followed by a mixing step (b),
steps (a) and (b) may be repeated at least once, with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result, and
step (a) may be repeated at least once and after step (b), steps (a) and (b) may be repeated at least once, with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result.

In the present disclosure, by repeating step (a) it should be understood that the process comprises at least one subsequent step (a) after step (a) wherein the harvested inocula obtained in step (a) are re-cultivated with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in step (a) depending on the intended result.

In the present disclosure, by repeating steps (a) and (b) it should be understood that the process comprises at least one subsequent step (a) followed by a subsequent step (b) after the step (b) wherein the expanded complex community of microorganisms obtained in the step (b) is re-cultivated in at least two bioreactors, with the same parameter such as pH, temperature, pressure, cultivation time, retention time, gassing conditions, redox potential, and combination thereof, preferably pH and/or retention time as previously defined, or with different parameter such as pH, temperature, pressure, cultivation time, retention time, gassing conditions, redox potential, and combination thereof, preferably pH and/or retention time as previously defined, as those previously used in the step (a) depending on the intended result, followed by a step (b).

The method of present disclosure may comprise a freezing, a thawing and/or a lyophilisation step as previously described between steps (a) and (b), between each or some repetitions of the step (a), and/or between each or some repetitions of steps (a) and (b).

In case of a complex community of microorganisms coming from at least two sources and/or donors, the samples of complex community of microorganisms of different sources and/or donors, may be mixed before step (a), after step (a), before of some or all repetitions of step (a) or after some or all repetitions of step (a). That is, the complex community of microorganisms coming from at least two sources and/or donors may be mixed:
before step (a) or before of some or all repetitions of step (a): in this case during step (a) at least one bioreactor comprises a mixture of a complex community of microorganisms coming from at least two sources and/or donors, or
after step (a) wherein step (a) has not been repeated: in this case each bioreactor comprises a complex community of microorganisms coming from a single source and/or donor.

Thus, in the method of the present disclosure, when the complex community comes from at least two sources and/or donors, the samples of complex community of microorganisms of different sources and/or donors may be mixed before step (a) and/or before the repetition of step (a) such that each bioreactor used in step (a) comprises different proportions of the complex community of microorganisms of at least one or different sources and/or donors, with the proviso that at least one bioreactor comprises a mixture of a complex community of microorganisms coming from at least two sources and/or donors.

All steps or some steps of the present disclosure may be carried out anaerobically.

Another object of the present disclosure relates to an expanded complex community of microorganisms obtained by the method of the present disclosure.

The expanded complex community of microorganisms may comprise at least three bacterial phyla selected from Bacteroidetes, Synergistetes, Firmicutes, Proteobacteria, Actinobacteria, Verrucomicrobia, Tenericutes and mixtures thereof.

The expanded complex community of microorganisms may comprise at least three bacterial phyla selected from Bacteroidetes, Synergistetes, Firmicutes, Proteobacteria, Actinobacteria, Verrucomicrobia, Tenericutes, Fusobacteria and mixtures thereof.

Preferably, the expanded complex community of microorganisms comprises Firmicutes, Bacteroidetes, Actinobacteria and Proteobacteria preferably with a sum of the relative abundances superior or equal to 80%, preferably superior or equal to 90%%, and more preferably superior or equal to 95%.

Preferably, the expanded complex community of microorganisms comprises:
Firmicutes at a relative abundance superior or equal to 30%, preferably superior or equal to 40%, and even more preferably superior or equal to 50%,
Bacteroidetes at a relative abundance inferior or equal to 50%, preferably inferior or equal to 40%, and even more preferably inferior or equal to 30%,
Actinobacteria at a relative abundance included between 1% and 20%, preferably between 2% and 15%, and even more preferably between 5% and 10%,
Proteobacteria at a relative abundance inferior or equal to 10%, preferably inferior or equal to 5%, and even more preferably between inferior or equal to 3%

Preferably, said phyla are coming from the complex community of microorganisms used in step a).

Preferably, the expanded complex community of microorganisms comprises at least 15 genera selected from the group comprising Clostridium, Eubacterium, Anaerostipes, Bacteroides, Bifidobacterium, Blautia, Butyricicoccus, Collinsella, Dialister, Dorea, Eisenbergiella, Escherichia-Shigella, Faecalibacterium, Fusicatenibacter, Hungatella, Lachnoclostridium, Parasutterella, Roseburia, Eubacterium, [Eubacterium] coprostanoligenes group, [Eubacterium] fissicatena group, [Ruminococcus] torques group, Allisonella, Bilophila, Christensenella, Christensenellaceae R-7 group, Clostridium, Clostridium sensu stricto 1, Coporococcus, Desulfovibrio, Enterococcus, Flavonifractor, Lachnospiraceae NK4A]36 group, Lachnospiraceae UCG-001, Lactobacillus, Parabacteroides, Peptostreptococcus, Romboutsia, Ruminoclostridium, Ruminococcaceae UCG-003, Ruminococcaceae UCG-O]3, Ruminococcaceae UCG-O]4, Ruminococcus, Subdoligranulum and Sutterella and mixtures thereof.

Preferably, said genera are coming from the complex community of microorganisms used in step a).

The expanded complex community of microorganisms may have an Operational Taxonomic Unit (OTU)-based richness superior or equal to 150, preferably superior or equal to 200, more preferably superior or equal to 275. OTUs are cluster of similar sequence variants of the 16S rDNA marker gene sequence. The OTU-based richness index represents the number of different OTUs found in the analysed expanded complex community of microorganisms. The OTU-based richness index may be calculated according to the method described in the example part. In particular, Genomic DNA may be extracted using the NucleoSpin Soil kit (Machery Nagel). A sequencing library targeting the V3-V4 region of the 16S rRNA gene may be constructed using the MyTaq HS-Mix 2×, Bioline, according to the manufacturer's instructions. Libraries may be then pooled in an equimolar mixture and sequenced in paired-end (2×300 bp) MiSeq V3 runs, Illumina. After amplicon merging using FLASH (FLASH:fast length adjustment of short reads to improve genome assemblies, Magoc and Salzberg, 2011, Bioinformatics. 2011 Nov. 1; 27(21):2957-63. doi: 10.1093/bioinformatics/btr507. Epub 2011 Sep 7.) and quality filtering using Trimmomatic (Trimmomatic: a flexible trimmer for Illumina sequence data, Bolger et al., 2014, Bioinformatics. 2014 Aug. 1; 30(15):2114-20. doi: 10.1093/bioinformatics/btu170. Epub 2014 Apr 1.), host sequence decontamination may be performed with Bowtie2 (Fast gapped-read alignment with Bowtie 2, Langmead et al., Nature Methods. 2012, 9:357-359.). Operational Taxonomic Unit (OTU) sequence clustering may be performed with an identity threshold of 97% using VSEARCH (VSEARCH: a versatile open source tool for metagenomics, Rognes et al., 2016, PeerJ. 2016 Oct. 18; 4:e2584. doi: 10.7717/peerj.2584. eCollection 2016.) and taxonomic profiling may then performed with the Silva SSU database Release 128.

Taxonomic and diversity analyses may be performed with R Statistical Software (R Core Team 2015, version 3.4.4) using vegan and phyloseq packages. For fair comparison, the sequence number of each sample may be randomly normalized to the same sequencing depth, i.e. 50000 amplicons per sample, and normalized by total bacteria count based on qPCR results. Diversity measures may correspond to the median value of 20 sub-samplings per sample.

The expanded complex community of microorganisms may have a richness at genus level superior or equal to 60, preferably superior or equal to 70 and even more preferably superior or equal to 75. Richness at genus level represents the number of different genera found in the analysed complex community of microorganisms.

The expanded complex community of microorganisms may have a Butycore superior or equal to 20%, preferably superior or equal to 30% and even more preferably superior or equal to 40%. The butycore represents the abundance of bacteria known to produce Butyrate. Bacteria known to produce Butyrate are selected from the group comprising Blautia, Faecalibacterium, Aiistipes, Eubacterium, Bifidobacterium, Ruminococcus, Clostridium, Coprococcus, Odoribacter, Roseburia, Holdemanella, Anaerostipes, Oscillibacter, Subdoligranulum and Butyrivibrio.

The expanded complex community of microorganisms may have a core superior or equal to 40%, preferably superior or equal to 50% and more preferably superior or equal to 60%. The core microbiota represents total abundance of following genera: Ruminococcus, Faecalibacterium, Dorea, Corprococcus, Blautia, Alistipes, Bacteroides, Subdoligranulum, Roseburia, Parabacteroides and Lachnospira.

The expanded complex community of microorganisms may have a “health index” superior or equal to 40%, preferably superior or equal to 50%, and more preferably superior or equal to 60%. The health index represents the sum of abundances of Lachnospiraceae, Ruminococcaceae, Clostridiaceae, Prevotellaceae and Erysipelotrichaceae.

The expanded complex community of microorganisms may have similarity between the complex community of microorganisms before and after its expansion superior or equal to 30%, preferably superior or equal to 70% and more preferably superior or equal to 90% as measured by the Jaccard index. A Jaccard index is a statistic tool used for gauging the similarity and diversity of sample sets.

The Jaccard coefficient measures similarity between two samples. The Jaccard index may be calculated at the genus or the family level. The Jaccard index is a tool well-known from the skilled person and is for example described in The longterm stability of the human gut microbiota, Faith, J. J., Guruge, J. L., Charbonneau, M., Subramanian, S., Seedorf, H., Goodman, A. L., et al. (2013), Science 341(6141), 1237439. doi: 10.1126/science.1237439.

In the next paragraphs, by “expanded complex community of microorganisms of the present disclosure” it will be understood an expanded complex community of microorganisms obtained by the method of the present disclosure optionally having the above-mentioned technical features.

Another object of the present disclosure relates to a pharmaceutical formulation comprising the expanded complex community of microorganisms of the present disclosure.

As used herein, the term “pharmaceutical composition” refers to any composition comprising the expanded complex community of microorganisms of the present disclosure and at least one pharmaceutically acceptable excipient.

As used herein, the term “pharmaceutically acceptable excipient” refers to a carrier medium which does not interfere with the effectiveness of the biological activity of the active ingredient(s) and which is not excessively toxic to the host at the concentration at which it is administered. Said excipients may be selected, depending on the pharmaceutical form and the desired method of administration, from the usual excipients known by a person skilled in the art.

The expanded complex community of microorganisms of the present disclosure or the pharmaceutical composition comprising said complex of the present disclosure may be administered by any of the accepted modes of administration, preferably by oral route, rectal route, gastroduodenal and skin (ointment).

Examples of oral formulations comprise but are not limited to, the formulations described in the patent application WO 2019/097030 A1.

The expanded complex community of microorganisms of the present disclosure or the pharmaceutical composition comprising said complex community of the present disclosure may be present under any suitable form for administration such as for example tablets, capsules, enema and suppository, formulated as a liquid, a suspension, a gel, a geltab, a semisolid, a tablet, a sachet, a lozenge, a capsule, an enteral formulation as an enema, or a skin formulation as an ointment.

Another object of the present disclosure relates to an expanded complex community of microorganisms of the present disclosure or the pharmaceutical composition comprising said complex community of the present disclosure for its use as a medicament.

The subject to be treated may be a human and/or an animal.

Another object of the present disclosure relates to an expanded complex community of microorganisms of the present disclosure or the pharmaceutical composition of the present disclosure for its use in microbiome ecosystem therapy such as autologous or allogeneic microbiome ecosystem therapy.

As used in the present disclosure, the term “microbiome ecosystem therapy” refers to administration of an expanded complex community of microorganisms obtained according the method of the present disclosure, at least one part or all the complex community of microorganisms cultivated in step (a) may have been extracted, partially extracted, partially isolated, isolated, partially separated, separated or not from its initial matrix or artificially synthesised or genetically modified. A microbiome ecosystem therapy may refer to the replacement of at least one part or all of a dysfunctional, damaged ecosystem with a fully functional and healthy ecosystem in a subject in need thereof.

Another object of the present disclosure relates to an expanded complex community of microorganisms of the present disclosure or the pharmaceutical composition of the present disclosure that it is under a form suitable for microbiome ecosystem therapy such as autologous or allogeneic microbiome ecosystem therapy.

Another object of the present disclosure relates to an expanded complex community of microorganisms of the present disclosure or the pharmaceutical composition comprising said complex community of the present disclosure for its use in the prevention and/or treatment of septicaemia, septic shock, infection such as Clostridioides difficile infection and associated diarrhoea (CDI), ulcerative colitis, inflammatory bowel disease (IBD), irritable bowel syndrome (IBS), idiopathic constipation, Coeliac disease, Crohn's disease, type I diabetes, type II diabetes, food allergies, solid cancers such as renal cell carcinoma, melanoma, colorectal or non-small cell lung cancer, liquid cancers such as leukemias or myelodysplastic syndrome, Graft-versus-host disease (GvHD) such as gastrointestinal acute GvHD, steroid-resistant GvHD or steroid-dependant GvHD, obesity and morbid obesity, autism, sclerosis, chronic vaginal infection such as cystitis and mycosis, bone and joint infections, Parkinson's disease, Alzheimer's disease, schizophrenia and bipolar disorders, gastrointestinal disorders such as intestinal inflammation, diarrhoea, traveller's diarrhoea, mucositis, abdominal pain and gastrointestinal bleeding, intestinal dysbiosis such as intestinal dysbiosis associated with cancer chemotherapy or immunotherapy, intensive care unit (ICU) related dysbiosis, alcoholic and non-alcoholic liver disease, treatment-induced inflammation such as treatment-induced gut inflammation, inflammation induced by anti-cancer therapy, hematologic disease, viral coronavirus infections, infectious or non-infectious complications resulting from an allogenic hematopoietic stem cell transplantation (allo-HSCT) such as steroid-refractory graft versus host disease (SR-aGvHD) following allogenic hematopoietic stem cell transplantation (allo-HSCT), carrying or infection by multi-resistant bacteria including Clostridium, Enterococci such as vancomycin-resistant enterococci (VRE) and Glycopeptide-resistant Enterococci (GRE), bacteria producing extended-spectrum beta-lactamases (ESBL), carbapenemase-producing Enterobacteriaceae (CPE), Emerging Bacteria Highly Resistant to Antibiotics (BHRe), and/or malignant hemopathy, and combination thereof.

In some of the abovementioned diseases, the expanded complex community of microorganisms of the present disclosure has a direct effect. In other diseases, the expanded complex community of microorganisms of the present disclosure is used in combination with drug-based therapy, immunotherapy, one or more immune checkpoint inhibitors such as those targeting PD-1, PD-L1, CD27, CD28, CD40, CD122, CD137, OX40, GITR, ICOS, A2AR, B7-H3, B7-H4, BTLA, CTLA-4, IDO, KIR, LAG3, NOX2, TIM-3, VISTA, SIGLEC7, therapy targeting tumor neoantigens, chemotherapy, radiation therapy and combination thereof, for example as an adjuvant increasing the effect of said therapy and/or to decrease the side effects induced by said therapy.

The present disclosure also relates to the use of the expanded complex community of microorganisms of the present disclosure or the pharmaceutical composition comprising said expanded complex community of microorganisms of the present disclosure for the manufacture of a medicament for the prevention and/or treatment of the above-mentioned diseases.

The present disclosure also relates to a method for the prevention and/or treatment of the above-mentioned diseases comprising a step of administrating to a subject the expanded complex community of microorganisms of the present disclosure or the pharmaceutical composition comprising said expanded complex community of microorganisms of the present disclosure.

Another object of the present disclosure relates to the expanded complex community of microorganisms of the present disclosure or the pharmaceutical composition comprising said expanded complex community of microorganisms of the present disclosure for its use in the prevention and/or treatment of the above-mentioned diseases in microbiome ecosystem therapy such as autologous or allogeneic microbiome ecosystem therapy.

The present disclosure also relates to the use of the expanded complex community of microorganisms of the present disclosure or the pharmaceutical composition comprising said expanded complex community of microorganisms of the present disclosure in microbiome ecosystem therapy such as autologous or allogeneic microbiome ecosystem therapy.

### BRIEF DESCRIPTION OF THE FIGURES

Results of example 1:

FIG. 1 shows the Taxonomy at phylum level of fermenters' mixes samples for runs 1, 2 and 3 in comparison to samples coming from single fermenters.

FIG. 2 shows the Richness at OTU level of fermenters' mixes samples for runs 1, 2 and 3 in comparison to samples coming from single fermenters.

FIG. 3 shows the conservation of OTUs and genera from baseline of fermenters' mixes samples for runs 1, 2 and 3.

FIG. 4 shows the butycore abundance of fermenters' mixes samples for runs 1, 2 and 3 in comparison to samples coming from single fermenters.

FIG. 5 shows the core microbiota abundance of fermenters' mixes samples for runs 1, 2 and 3 in comparison to samples coming from single fermenters.

FIG. 6 shows the health index abundance of fermenters' mixes samples for runs 1, 2 and 3 in comparison to samples coming from single fermenters.

FIG. 7 shows the β-diversity represented by Jaccard index similarity compared to baseline of fermenters' mixes samples for runs 1, 2 and 3.

Results of Example 2:

FIGS. 8A and 8B show the Taxonomy at phylum level of the mixes.

FIGS. 9A and 9B show the Richness at genus level of the mixes.

FIGS. 10A and 10B show the conservation of genera from baseline of the mixes.

FIGS. 11A and 11B show the core microbiota abundance of the mixes.

FIGS. 12A and 12B show the butycore abundance of the mixes.

FIGS. 13A and 13B show the health index abundance of the mixes.

FIGS. 14A and 14B show the β-diversity represented by Jaccard index similarity compared to baseline of the mixes.

FIG. 15: Protocol for preparing Mix 28 to 31

Results of example 3:

FIG. 16 shows the taxonomy at phylum level of fermenters' mixes coming from different retention time conditions in comparison to samples coming from single fermenters.

FIG. 17 shows the richness at genus level of fermenters' mixes coming from different retention time conditions in comparison to samples coming from single fermenters.

FIG. 18 shows the butycore abundance of fermenters' mixes coming from different retention time conditions in comparison to samples coming from single fermenters.

FIG. 19 shows the core microbiota abundance of fermenters' mixes coming from different retention time conditions in comparison to samples coming from single fermenters.

FIG. 20 shows the health index abundance of fermenters' mixes coming from different retention time conditions in comparison to samples coming from single fermenters.

FIG. 21 shows the β-diversity represented by Jaccard index similarity of fermenters' mixes at genus level coming from different retention time conditions in comparison to samples coming from single fermenters.

### Results of Example 4

FIG. 22 shows the taxonomy at phylum level of the mixes for run 1, 2, 3 and 4.

FIG. 23 shows the butycore abundance of the mixes for run 1, 2, 3 and 4.

FIG. 24 shows the core microbiota abundance of the mixes for run 1, 2, 3 and 4.

### EXAMPLES

### Example 1: pH Impact on Microbiota Fermentation

Scope of the Experiment

The purpose of the experiment was to investigate the effect of three different pH on microbiota fermentation and to establish if mixing microbiota stabilized at different pH enables to better correspond to the initial microbiota taxonomic profile.

Protocol
Fermentation System

Mobius CellReady 3 L disposable bioreactors were used. Mobius bioreactors are manufactured by the company Millipore.

Bioreactors are monitored by My-Control controllers. Controllers are manufactured by the company Applikon Biotechnology (Netherlands). These systems integrate the main parameters of the in vivo human colonic environment, such as pH, body temperature, supply of ileal effluents, retention time, anaerobiosis maintained by the activity of resident microbiota (Dynamic In Vitro Models of the Human Gastrointestinal Tract as Relevant Tools to Assess the Survival of Probiotic Strains and Their Interactions with Gut Microbiota, Cordonnier et al., 2015, Microorganisms. 2015 December; 3(4): 725-745).

The fermentation parameters were:
Reactional volume: 1 L
Set temperature: 37° C.
Regulate pH: 5.3; 6.3 or 7.3 (each bioreactor/fermenter having a different pH set point)
Retention time: 24 h

Medium's composition is as close as possible to the one of the ileal effluents (Dynamic In Vitro Models of the Human Gastrointestinal Tract as Relevant Tools to Assess the Survival of Probiotic Strains and Their Interactions with Gut Microbiota, Cordonnier et al., 2015, Microorganisms. 2015 Dec; 3(4): 725-745) and each component is food or pharma grade.

A 100 μL of antifoam agent (Y-30 Emulsion from Sigma-Aldrich referred as A5758) was added at the beginning of microbiota culture and medium's pH was adjusted at 5.3; 6.3 and 7.3 respectively according to the fermenter set points.
Human Faecal Microbiota Sourcing and Preparation
For each run, each bioreactor was inoculated with a frozen faeces sample, coming from the same donor at a concentration of 1 g/L. Three different donors were tested throughout the three runs of three fermenters.
Faeces were prepared according to the methods described in the international applications WO2016/170285 A1, WO2017/103550 A1 or WO2019/171012 A1. In particular, samples were collected, diluted in the diluent described in WO2016/170285 A1 and WO2017/103550 A1 (that is 5% trehalose and 15% maltodextrin) and stored at −80° C. until use.

Runs Performed

Three runs in three fermenters were performed:
Run 1: Donor 1
Run 2: Donor 2
Run 3: Donor 3

For each run, three fermentations were carried on in parallel in three different fermenters. The difference between these three fermenters was the pH set point which was set to 5.3; 6.3 and 7.3 respectively according to the fermenter.
Analysis Performed
16S rDNA sequencing:
   Mixes of the three pH conditions in different proportions according to table 1 were performed for 16S rDNA sequencing.

TABLE 1

| | | Bioreactor | Bioreactor | Bioreactor |
|---|---|---|---|---|
| | | having a pH | having a pH set | having a pH set |
| Name of the | | set point of | point of 6.3 | point of 7.3 |
| mix | Percentage | 5.3 (F1) | (F2) | (F3) |
| MIX 1 | 33.34%/33.33%/ | 333 μl | 333 μl | 333 μl |
| | 33.33% | | | |
| MIX 2 | 60%/30%/10% | 600 μl | 300 μl | 100 μl |
| MIX 3 | 10%/60%/30% | 100 μl | 600 μl | 300 μl |
| MIX 4 | 30%/10%/60% | 300 μl | 100 μl | 600 μl |

Sequencing was performed by Eurofins Genomics (Ebersberg, Germany). Genomic DNA was extracted using the NucleoSpin Soil kit (Machery Nagel). A sequencing library targeting the V3-V4 region of the 16S rRNA gene was constructed for each sample using the MyTaq HS-Mix 2X, Bioline, according to the manufacturer's instructions. Libraries were then pooled in an equimolar mixture and sequenced in paired-end (2×300 bp) MiSeq V3 runs, Illumina.

After amplicon merging using FLASH (FLASH: fast length adjustment of short reads to improve genome assemblies, Magoc and Salzberg, 2011, Bioinformatics. 2011 Nov. 1; 27(21):2957-63. doi: 10.1093/bioinformatics/btr507. Epub 2011 Sep 7.) and quality filtering using Trimmomatic (Trimmomatic: a flexible trimmer for Illumina sequence data, Bolger et al., 2014, Bioinformatics. 2014 Aug. 1; 30(15):2114-20. doi: 10.1093/bioinformatics/btul70. Epub 2014 Apr 1.), host sequence decontamination was performed with Bowtie2 (Fast gapped-read alignment with Bowtie 2, Langmead et al., Nature Methods. 2012, 9:357-359.). Operational Taxonomic Unit (OTU) sequence clustering was performed with an identity threshold of 97% using VSEARCH (VSEARCH: a versatile open source tool for metagenomics, Rognes et al., 2016, PeerJ. 2016 Oct. 18; 4:e2584. doi: 10.7717/peerj.2584. eCollection 2016.) and taxonomic profiling was then performed with the Silva SSU database Release 128.

Taxonomic and diversity analyses were performed with R Statistical Software (R Core Team 2015, version 3.4.4) using vegan and phyloseq packages. For fair comparison, the sequence number of each sample was randomly normalized to the same sequencing depth, i.e. 50000 amplicons per sample, and normalized by total bacteria count based on qPCR results. Diversity measures correspond to the median value of 20 sub-samplings per sample.
Flow Cytometry Analysis:

For flow cytometry analysis, samples were labelled with a live/dead kit and analyzed on a BD™ LSR II flow cytometer.

Results

Mixes of the Three pH Conditions in Different Proportions:

This part concerns the 16S results for fermenters' samples mixed at different days of fermentation (at day 2, at day 4, at day 8 and at day 14), in different proportions according to table 1 above.
16S for mixes:
Taxonomic composition at Phylum level:

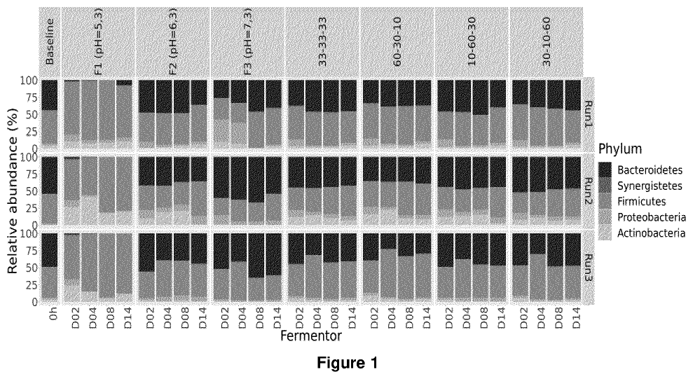

Results are shown on FIG. 1.

As shown on FIG. 1, no matter the proportions are, there is no difference in final composition. Nonetheless, composition is normalized and is closed to baseline composition.
Richness at OTU level:

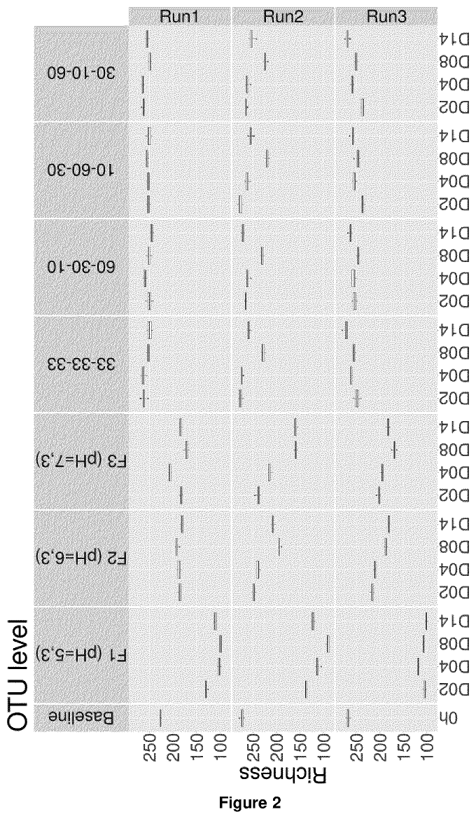

Results are shown on FIG. 2.

Here again, as shown on FIG. 2, richness is increased and normalized when at least two fermenters samples are mixed. This can be explained by the fact that at least two pH conditions lead to a different community of microorganisms, increasing the richness.
Origin of OTU's:

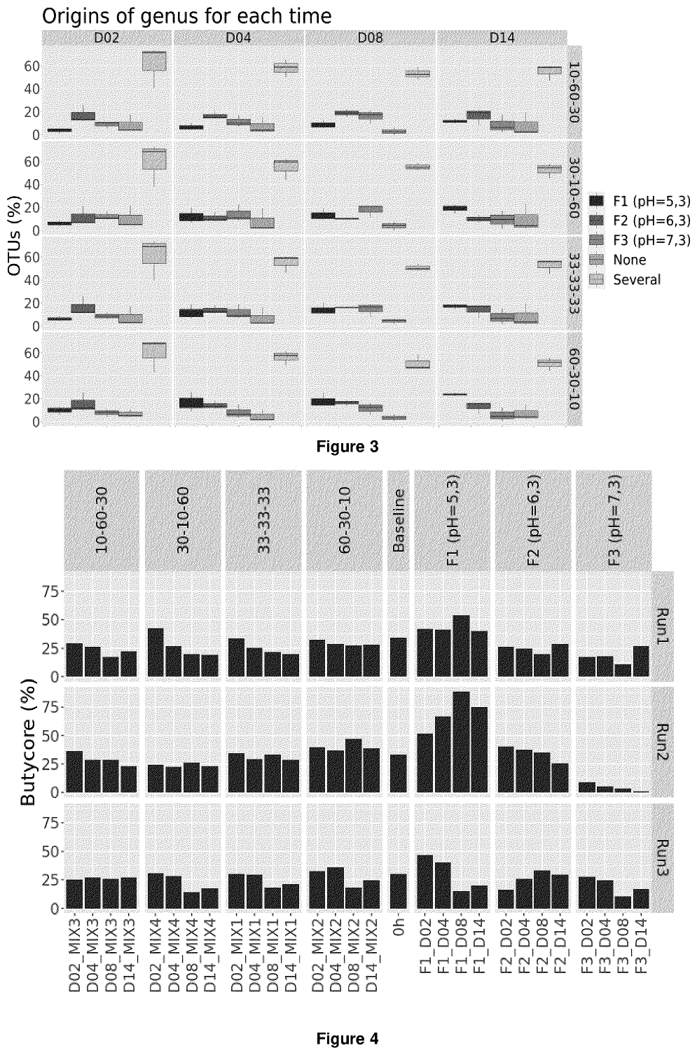

Results are shown on FIG. 3.

FIG. 3 shows that in all mixes, the majority of identified OTUs were also present in at least two fermenters. It also shows that each fermenter brings its own OTUs in the mix, meaning that at least two pH conditions are useful. Also, proportion effect is visible in this figure since the number of OTUs belonging to the fermenter present in the higher quantity brings generally a higher percentage of OTUs than the two others. For the 33-33-33 (more specifically 33,34-33,33-33,33) proportion, each fermenter brings almost the same amount of OTUs.

Surprisingly, there is also a small amount of OTUs coming from none of the fermenters probably due to OTUs present at very low abundances and not detectable in the beginning of the fermentation.

These OTUs could have increased in the fermentation conditions to be then detectable.
Butycore:

Butycore represents the abundance of bacteria known to produce Butyrate.

Results are shown on FIG. 4.

As shown on FIG. 4, when samples of fermenters are mixed, butycore is normalized. A small effect of proportion is observed for Run 2 mix 60-30-10 and Run 2 mix 30-10-60. In the first case, pH 5.3 fermenter (F1) presents the highest bacteria proportion, increasing the butycore compared to other mixes and being higher than baseline, since its single value is high (around 70% after day 4). In the second case, pH 8.3 fermenter (F3) presents the higher proportion, decreasing the butycore compared to other mixes, since its single value is low (less than 10% after day 2).
Core microbiota:

Core microbiota represents the total abundance of following genera: Ruminococcus, Faecalibacterium, Dorea, Corprococcus, Blautia, Alistipes, Bacteroides, Subdoligranulum, Roseburia, Parabacteroides and Lachnospira.

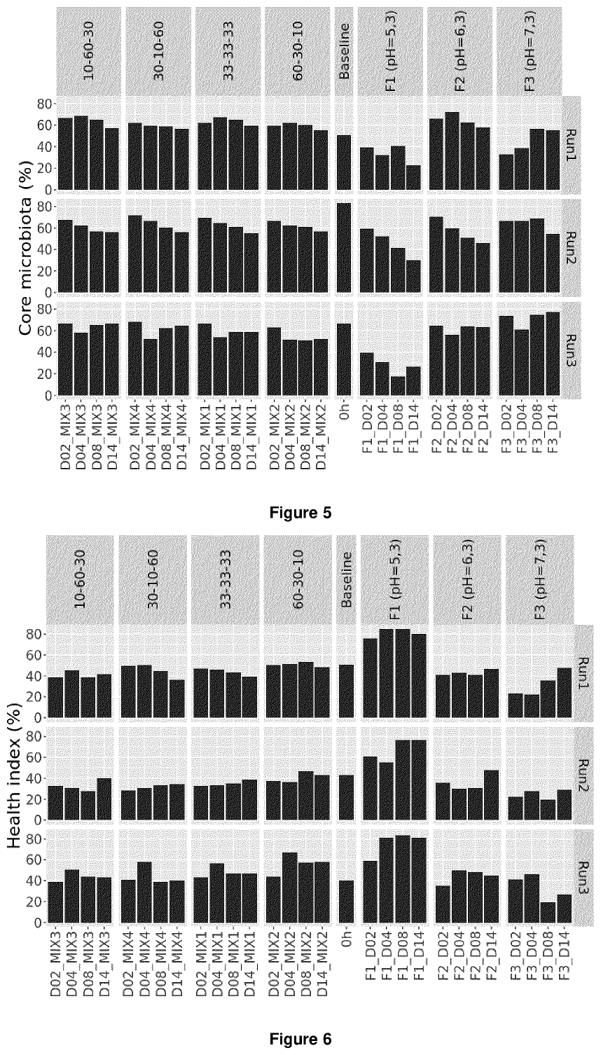

Results are shown on FIG. 5.

As shown on FIG. 5, core microbiota is also normalized to higher or similar values compared to baseline. No difference is observed between proportions. It seems to be a balance between all fermenters.
Health index:

Health index represents the sum of abundances of Lachnospiraceae, Ruminococcaceae, Clostridiaceae, Prevotellaceae and Erysipelotrichaceae.

Results are shown on FIG. 6.

As shown on FIG. 6, as other variables, health index is normalized when fermenters are mixed.

Values are similar to baseline ones. No significant difference is observed between proportions (except a slight effect of pH 5.3 (F1) when comparing mix 10-60-30 to mix 60-30-10 for all runs).
β-diversity, Jaccard similarity with baseline:

A Jaccard index is a statistic tool used for gauging the similarity and diversity of sample sets.

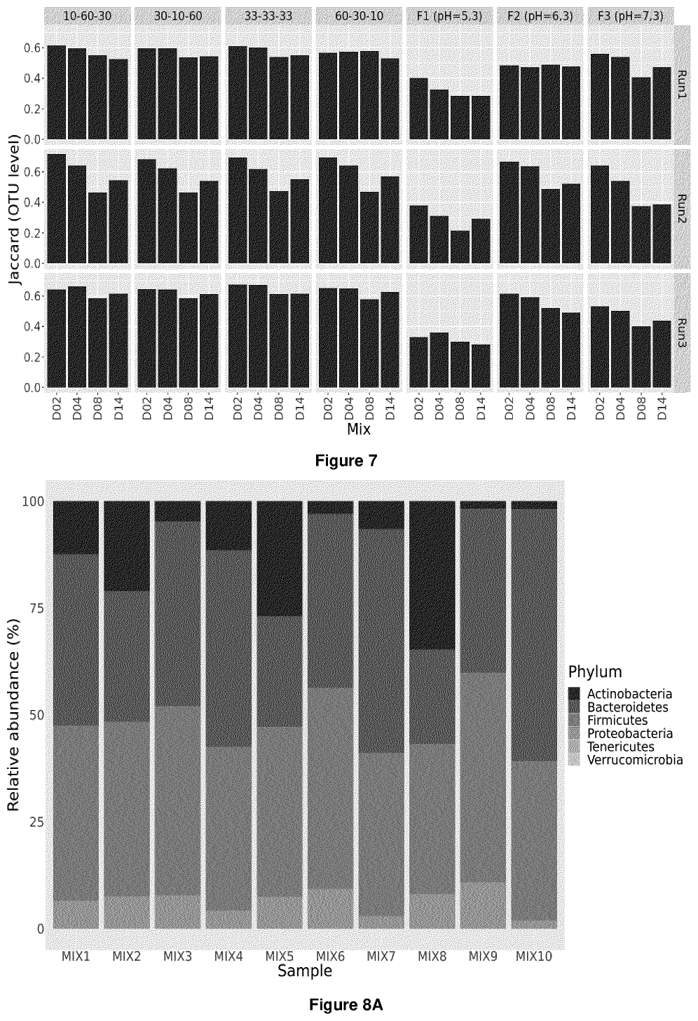

Results are shown on FIG. 7.

As shown on FIG. 7, Jaccard index is normalized when fermenters are mixed. At day 2 (D02) and day 4 (D04) Jaccard index of mixes is close to those of single fermenters after 4 h of fermentation (not shown), hence to baseline, since at 4h Jaccard index was around 70%. At day 8 (D08) and day 14 (D14), index decreases a bit but is almost always higher than single fermenters values (not shown).

### Discussion and Conclusion

Mixes of the three pH conditions in different proportions:

Results for mixes show mostly that at OTU level, no differences were observed between fermenters proportions and day of sampling. For instance, mixing fermenters allowed a normalization of the results and each index gets closer to baseline values when fermenters are mixed. It was the case for taxonomy at OTU, for butycore, core microbiota, health index, richness and β-diversity.

In particular, the majority of identified OTUs were present in at least two fermenters and each of the at least two fermenters brings its own OTUs in the mix, meaning that at least two pH conditions are useful.

As a conclusion, we have seen that working in at least two different pH brings OTUs of interest. Also, when at least two pH are combined, each variable gets closer to baseline values. That is, at least two pH conditions are important and will be kept for further experiments.

Example 2: Use of prediction tool, to mix several microbiota coming from in vitro fermentations

Scope of the Experiment

The tool used in this example allows the prediction of a profile obtained by mimicking the mix of several microbiota samples in silico. In this experiment the tool is used to mix 2, 3 or 4 sample profiles from in vitro fermentations. These samples are issued from fermentations performed with same or different donors and/or with same or different pH set point(s). The samples are also mixed in different proportions.

Protocol

Mixes

Tested proportions for the different mixes are presented hereunder.
MIX 1 to 7 are built with profiles, coming from three fermenters inoculated with a microbiota coming from the same donor, but with three different pH set points (5.3; 6.3 and 7.3) and MIX 8 to 10 are built with profiles coming from one fermenter inoculated with a microbiota coming from the same donor and with a single pH set point as comparison purposes:

TABLE 2

| Name of the | | | | |
|---|---|---|---|---|
| mix | pH 5.3 | pH 6.3 | pH 7.3 | Inoculum |
| MIX 0 | | | | 100 |
| MIX 1 | 33.34 | 33.33 | 33.33 | |
| MIX 2 | 60 | 30 | 10 | |
| MIX 3 | 10 | 60 | 30 | |
| MIX 4 | 30 | 10 | 60 | |
| MIX 5 | 80 | 15 | 5 | |
| MIX 6 | 5 | 80 | 15 | |
| MIX 7 | 15 | 5 | 80 | |
| MIX 8 | 100 | 0 | 0 | |
| MIX 9 | 0 | 100 | 0 | |
| MIX 10 | 0 | 0 | 100 | |

Mix 11 to 27 are built with two microbiota profiles, coming from two fermenters inoculated with microbiota coming from the same donor, with two different pH set points (5.3; 6.3 or 7.3):

TABLE 3

| Name of the | | | |
|---|---|---|---|
| mix | pH 5.3 | pH 6.3 | pH 7.3 |
| MIX 11 | 50 | 50 | |
| MIX 12 | | 50 | 50 |
| MIX 13 | 50 | | 50 |
| MIX 14 | 90 | 10 | |
| MIX 15 | 10 | 90 | |
| MIX 16 | | 90 | 10 |
| MIX 17 | | 10 | 90 |
| MIX 18 | 90 | | 10 |
| MIX 19 | 10 | | 90 |
| MIX 20 | 2 | 98 | |
| MIX 21 | 2 | | 98 |
| MIX 22 | 70 | 30 | |
| MIX 23 | 30 | 70 | |
| MIX 24 | | 70 | 30 |
| MIX 25 | | 30 | 70 |
| MIX 26 | 70 | | 30 |
| MIX 27 | 30 | | 70 |

Mix 28 to 31 are built with four microbiota profiles, coming from fermenters inoculated with microbiota coming from different donors, with different pH set points (5.3; 6.3 and/or 7.3) according to FIG. 15.

Mixes have been identified as seen in previous table: MIX 1 to 31.

Analysis Performed

Mixes are predicted on the prediction tool platform. The obtained .csv files summarizing the taxonomic composition of the predicted pools are exported, and analysed using bioinformatic tools in order to compute the following metrics:
Taxonomic composition at phylum level (Bacteroidetes, Firmicutes, Proteobacteria, . . . )
Richness (Genus level)
Conservation of genera found in baseline
Core microbiota
Butycore
Health index I2
β-diversity, Jaccard similarity with baseline

### Results

Taxonomic composition at the Phylum level

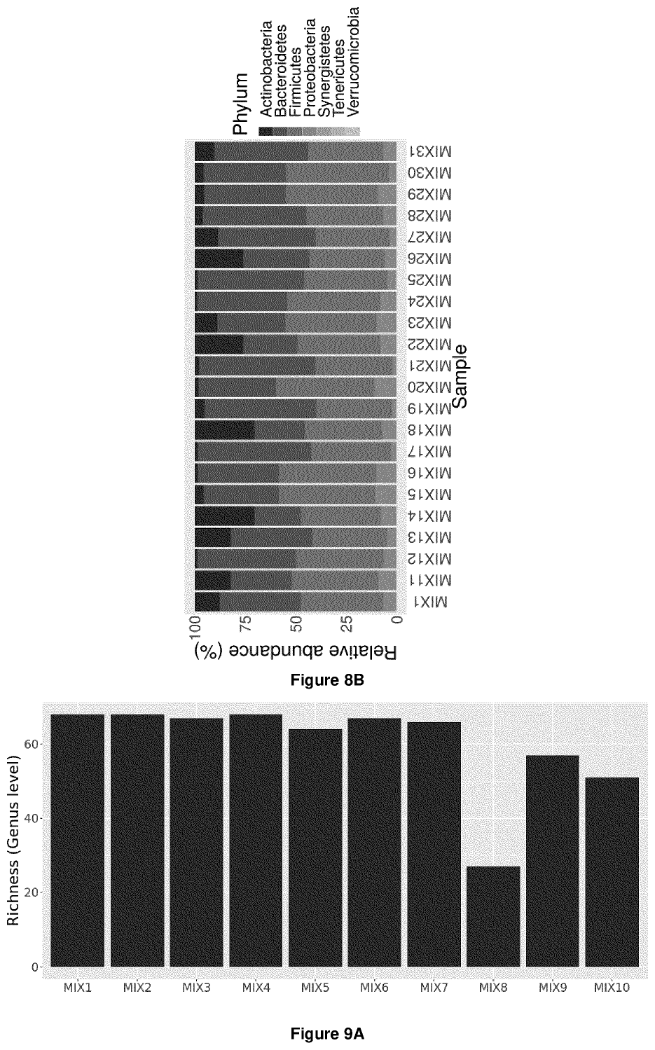

Results are shown on FIG. 8.

MIX8, MIX9 and MIX10 corresponds to samples coming from the first, second and third fermenter. As seen in Example 1, the sample coming from pH 5.3 fermenter (MIX8) has a composition with a higher abundance of Actinobacteria and a lower abundance of Bacteroidetes compared to samples coming from pH 6.3 and 7.3 fermenters (MIX9 and MIX10 respectively). Samples coming from pH 6.3 and pH 7.3 fermenters (MIX9 and MIX 10 respectively) are similar, excepted that there is a higher abundance of Proteobacteria in the sample coming from pH 6.3 fermenter (MIX 9).

Then, excepted for mixes in which a high proportion of sample coming from pH 5.3 fermenter is present (MIX2, MIX5, MIX8, MIX11, MIX13, MIX14, MIX18, MIX22 and MIX26), it can be seen that no matter the number of microbiota and no matter if pH is different, the profiles at phylum level are similar for all mixes.

Richness (Genus Level):

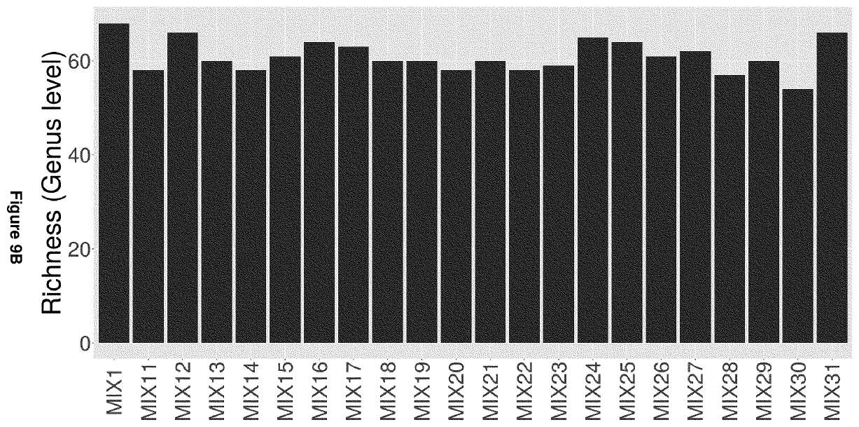

Results are shown on FIG. 9.

When microbiota are considered alone (MIX8, MIX9 and MIX10) the corresponding richness is lower than the richness of other mixes. All mixes present a richness that is similar and close to the initial sample (baseline—not shown) for MIX1 to MIX7 and to MIX1 for MIX11 to MIX31.

Conservation of genera found in baseline:

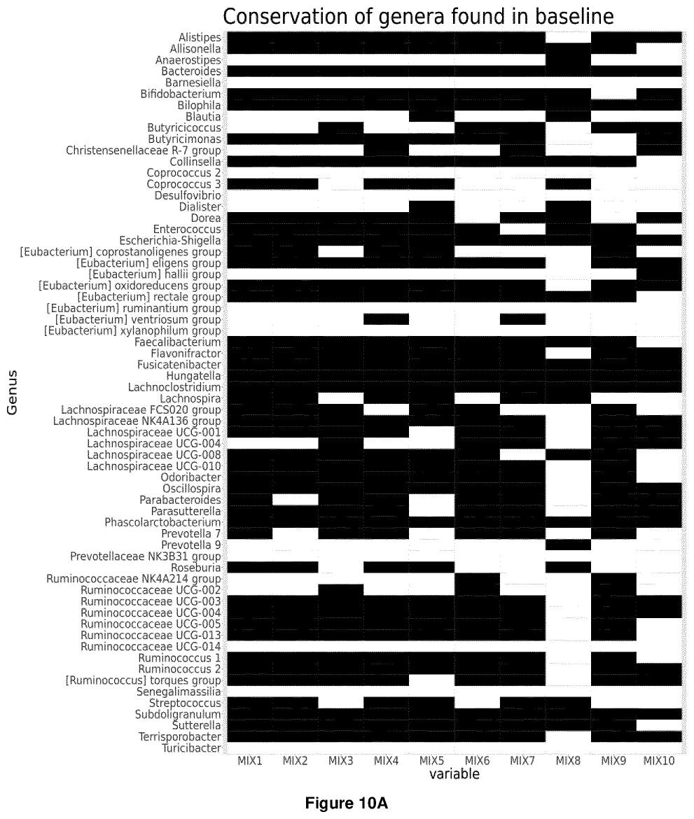

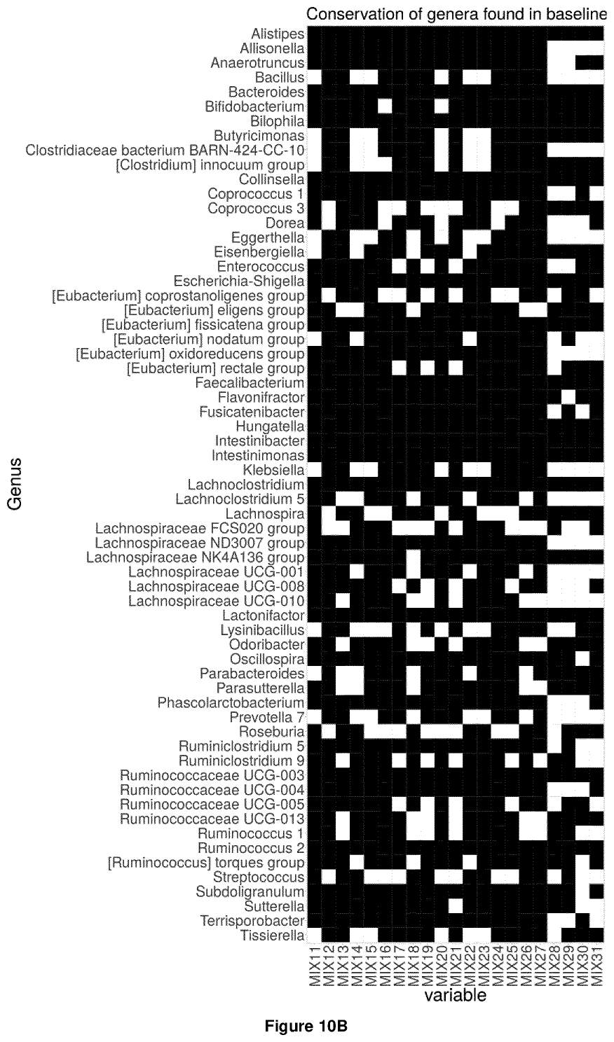

Results are shown on FIG. 10.

Here, the number of genera in MIX1 to MIX10 has been compared to the initial sample (baseline—not shown). The number of genera in MIX11 to MIX31 has been compared to MIX1.

That is, the initial sample is the baseline for MIX1 to MIX10 and MIX1 is the baseline for MIX11 to MIX31.

When comparing MIX 1 to 7 corresponding to mixes of samples coming from three fermenters with three different pH set points to samples coming from single fermenters (MIX8 to MIX10), it can be seen that mixing at least two microbiota as seen in example 1 allows keeping more genera than the use of single fermenters (MIX8 to MIX10). MIX 1 and MIX4 presents the higher number of genus, they correspond to 33/33/33 (more specifically 33,34/33,33/33,33), 60/30/10, 10/60/30 and 30/10/60 proportions respectively. Looking at genera belonging to core microbiota, it can be seen that no genus is lost totally when mixing. Some genera like Blautia or Roseburia are hard to keep in mixes but their presences are only proportion dependent. For the first one, since it is only found in samples coming from pH 5.3 fermenters it is only found in mixes containing a high proportion of sample coming from this fermenter. For the second one, similarly, it is only found in sample coming from pH 5.3 fermenters therefore when the mix contains a low proportion of sample coming from pH 5.3 fermenters, the genera is lost in the mix.

Concerning MIX 11 to 31, it can be seen that mixing only two microbiota (MIX11 to MIX27) also allows keeping a high number of genera and most of core microbiota genera are maintained in all mixes. For MIX 28 to 31, which can be compared to a mix that contains microbiota coming from different donors different from the donor of MIX1, the number of lost genera is, as expected, higher than for other mixes. Nonetheless, the number of lost genera in MIX 28 to 31 is similar to the one of sample mixes with three microbiota coming from fermenters with three different pH set points (MIX1 to 7).
Core microbiota

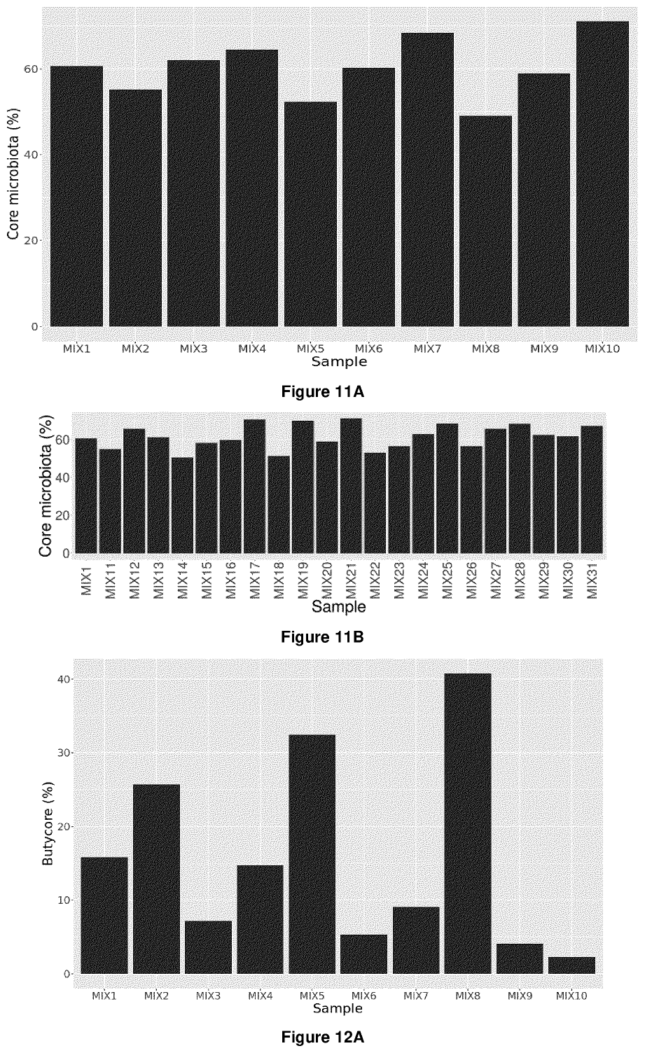

Results are illustrated in FIG. 11.

Core microbiota represents total abundance of following genera: Ruminococcus, Faecalibacterium, Dorea, Coprococcus, Blautia, Alistipes, Bacteroides, Subdoligranulum, Roseburia, Parabacteroides and Lachnospira.

As seen on FIG. 11, Core microbiota is similar for all mixes and a bit lower for mixes in which the proportion of sample coming from pH 5.3 fermenter is high (since core microbiota values are low in samples coming from pH 5.3 fermenter only). No real difference is observed between mix of three, two and four microbiota and values are always close to 60%.
Butycore

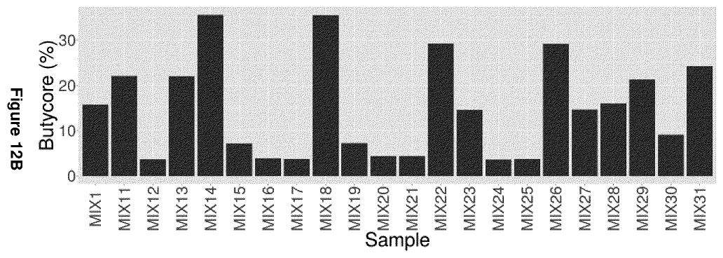

Results are shown on FIG. 12.

Butycore represents abundance of bacteria known to produce Butyrate. As seen in example 1, butycore index is high for pH 5.3 fermenters. It is also the case for MIX8 which corresponds to a sample coming from pH 5.3 fermenter alone. So, when mixes comprises samples coming from pH 5.3 fermenter butycore index is higher in comparison to mixes that does not comprise samples coming from pH 5.3 fermenter. Interestingly, when mixes comprise samples coming from three different donors with no pH modification, butycore is high (MIX28). Butycore is then higher for mixes comprising samples coming from pH 5.3 fermenters and/or coming from several donors.
Health index 12

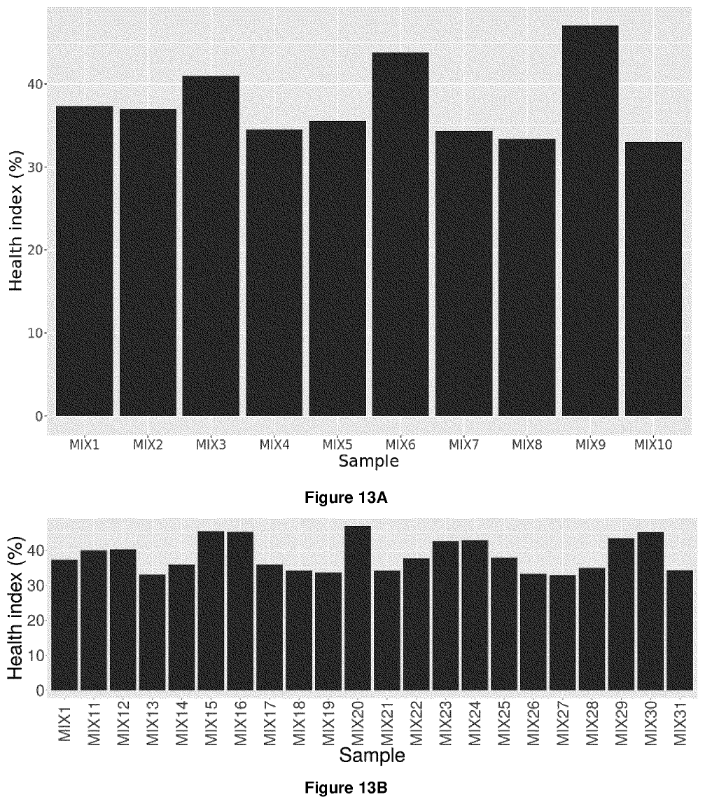

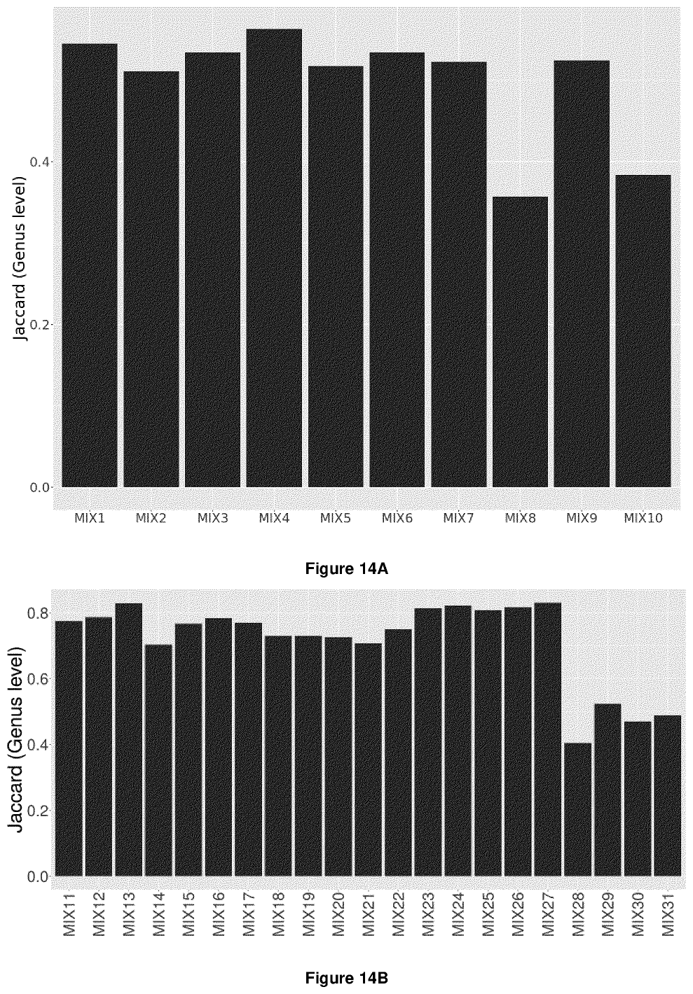

Results are shown on FIG. 13.

Health index represents the sum of abundances of Lachnospiraceae, Ruminococcaceae, Clostridiaceae, Prevotellaceae and Erysipelotrichaceae.

FIG. 13 shows that no matter the number of donor and no matter the number of pH condition, Health index is always between 30 and 45%. The percentage is even higher for mixes comprising a high proportion of samples coming from pH 6.3 fermenter. It was expected since the sample coming from pH 6.3 fermenter alone shows high Health index values.
β-diversity, Jaccard similarity with baseline

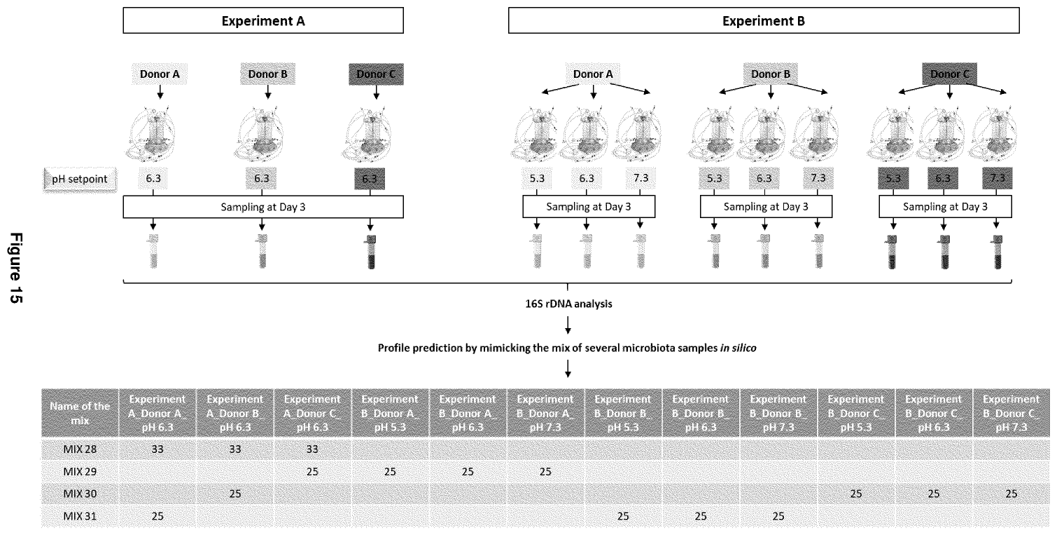

Results are shown on FIG. 14.

Similarity for mixes comprising samples coming from single fermenters (MIX 8 to 10) with the initial sample (baseline—not shown) is lower than mixes comprising samples coming from different fermenters (MIX1 to 7 and 11 to 27) and is even higher for mixes comprising samples coming from only two fermenters (MIX11 to MIX27).

As expected, similarity between MIX1 and MIX28 to 31 is lower since initial donor is not the same.

Nonetheless, value is still close to 40%.

### Discussion and Conclusion

In this experiment it has been demonstrated that mixing fermenters inoculated with a microbiota coming from a single donor and with at least two different pH set points had an impact on several indexes by improving them compared to single fermenters conditions, as seen in example 1. Moreover, it has been seen that pH 5.3 condition had an impact on these indexes by increasing or decreasing values. This condition is thus important in mixes and should be kept in order to influence final microbiota composition.

The tool results also showed that with only 2 pH conditions indexes are better in comparison to those obtained with single fermenters (1 pH condition). And more interestingly, mixing four microbiota coming from several donors and/or at several pH set points allows getting closer from baseline values than with single fermenters (1 pH condition).

As a conclusion, this example showed that the culture and mix of 2, 3 and 4 microbiota is possible and is required compared to the use of single fermenters (1 pH condition) to get closer to initial profile and mixing with different proportions can allow influencing final composition of the microbiota.

Culture can work with at least 2 microbiota (at least 2 pH conditions) but it can be easily imagined to enrich this mixed microbiota by adding more pH conditions and/or more initial donors and also by changing proportion of each microbiota since it will allow maintaining as many genera as possible and get even closer to the initial microbiota before fermentation.

### Example 3: Retention time impact on microbiota fermentation

### Scope of the Experiment

The purpose of the experiment was to investigate the effect of four retention time conditions on microbiota fermentation.

Protocol

The protocol was identical to example 1 at the exception that fermentation parameters and flow cytometry analysis differed.
Fermentation parameters
The fermentation parameters were:
Reactional volume: 1 L
Set temperature: 37° C.
Regulate pH: 6.3
Retention time: 12h, 24 h, 48h, 96h (each bioreactor/fermenter having a different retention time)

Only one human fecal microbiota, sourced as described in example 1, was used.
Flow cytometry analysis

For flow cytometry analysis, samples were labelled with a live/dead kit and analyzed on a Guava® easyCyte™ 5HT flow cytometer.
Run performed
Four fermentations were performed:
RT1: retention time of 12h
RT2: retention time of 24 h
RT3: retention time of 48h
RT4: retention time of 96h

Four fermentations were carried out in parallel in four different fermenters. The difference between these four fermenters was the retention time set point which was set to 12h, 24 h, 48h and 96h, respectively.
Mix performed

Mixes were performed using an in sillico prediction tool. The Error! Reference source not found. presents the proportion used in the prediction tool.

TABLE 4
Proportion of single fermenters in mixes performed in sillico

| Mix | | | | |
|---|---|---|---|---|
| percentages | RT1 | RT2 | RT3 | RT4 |
| MIX 1 | 25 | 25 | 25 | 25 |
| MIX 2 | 10 | 20 | 30 | 40 |
| MIX 3 | 20 | 30 | 40 | 10 |
| MIX 4 | 30 | 40 | 10 | 20 |
| MIX 5 | 40 | 10 | 20 | 30 |
| MIX 6 | 10 | 20 | 20 | 50 |
| MIX 7 | 20 | 20 | 50 | 10 |
| MIX 8 | 20 | 50 | 10 | 20 |
| MIX 9 | 50 | 10 | 20 | 20 |
| MIX 10 | 10 | 30 | 60 | |
| MIX 11 | 30 | 60 | 60 | |
| MIX 12 | 60 | 10 | 30 | |
| MIX 13 | 34 | 33 | 33 | |

Results

Taxonomic Composition at the Phylum Level

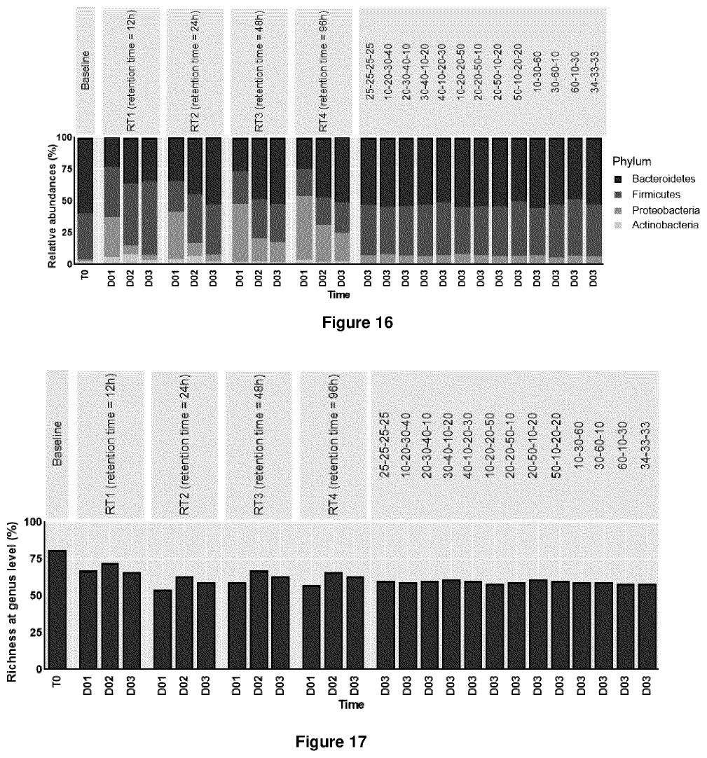

Results are shown on FIG. 16. As shown on FIG. 16, differences are observed in composition at D03 depending on the retention time used. More Firmicutes are obtained in the RT1 condition and more Proteobacteria are obtained in the RT4 condition. No matter the proportions are, there is no difference in final composition. Nonetheless, composition is normalized and is closed to baseline composition.

Richness at Genus Level

Results are shown on FIG. 17. As shown on FIG. 17, some differences are observed over fermentation time. At D03, richness indexes were similar whatever the retention time condition. When samples of fermenters are mixed, the richness is normalized, whatever the proportions.

Butycore

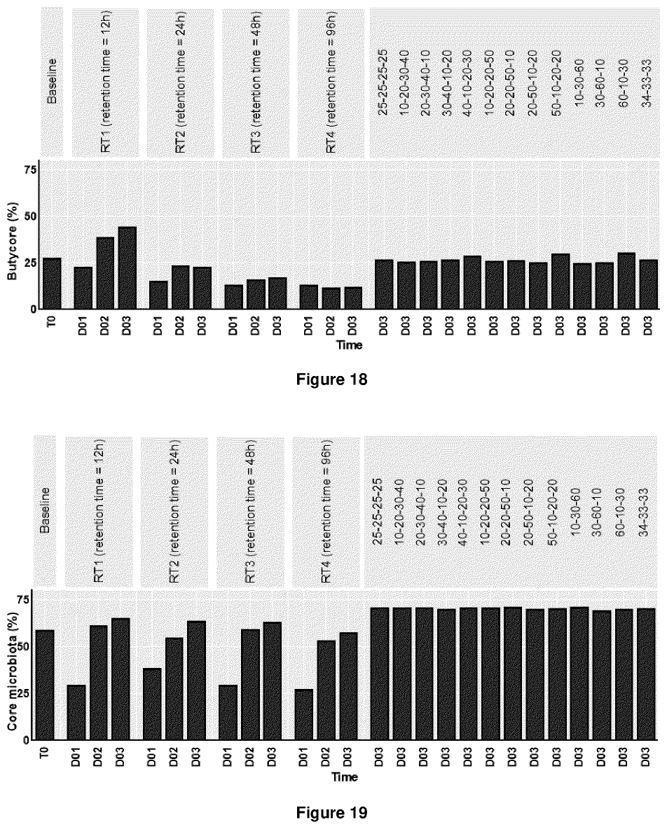

Butycore represents the abundance of bacteria known to produce butyrate. Results are shown in FIG. 18.

As shown on FIG. 18, already at D01, the butycore index is higher for RT1 condition. The RT4 condition is the least favorable for the butycore whatever the fermentation time. The RT1 condition allowed to obtain the highest butycore score even higher compared to the baseline. When samples of fermenters are mixed, butycore is normalized, whatever the proportions except for mixes where RT1 is added in higher proportion.
Core microbiota

Core microbiota represents the total abundance of following genera: Ruminococcus, Faecalibacterium, Dorea, Corprococcus, Blautia, Alistipes, Bacteroides, Subdoligranulum, Roseburia, Parabacteroides and Lachnospira. Results are shown on FIG. 19.

As shown on FIG. 19, whatever the retention time used, the core microbiota index increased over time to reach similar abundance at D03. Values are similar to baseline one. No difference is observed between proportions.
Health index

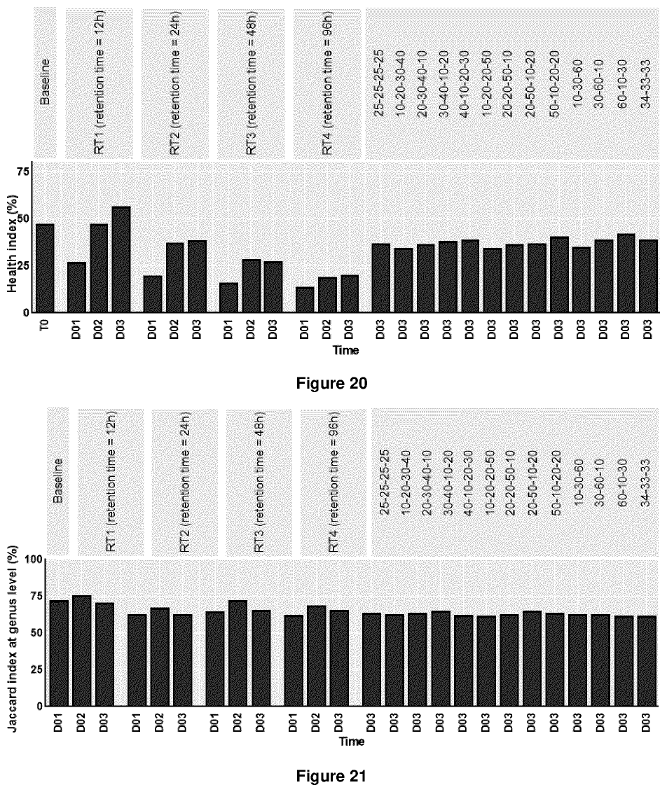

Health index represents the sum of abundances of Lachnospiraceae, Ruminococcaceae, Clostridiaceae, Prevotellaceae and Erysipelotrichaceae. Results are shown on FIG. 20.

As shown on FIG. 20, whatever retention times, the health index increased from D01 to D02. RT1 reached a higher health index at D03 compared to other retention time conditions. Health index tended to decrease with higher retention times. As other variables, health index is normalized when fermenters are mixed. No major differences are observed except with the mixes where RT1 is added in higher proportion.
β-diversity, Jaccard similarity with baseline

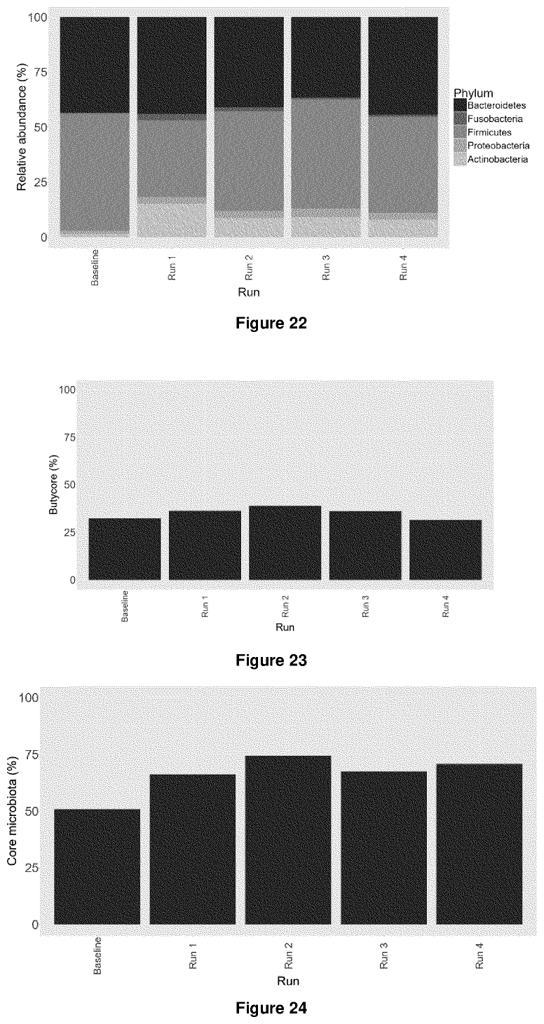

A Jaccard index is a statistic tool used for gauging the similarity and diversity of sample sets. Results are presented in FIG. 21.

As shown on FIG. 21, Jaccard index of each retention time condition is similar. Jaccard index of mixes is close to those of single fermenters after 72h of fermentation. No major differences are observed whatever proportion of mixes.

Discussion and Conclusion

Results show that retention time can influence development of different phyla and therefore different genera. The lowest retention time favored genera of interest that composed the core microbiota and butycore. Besides, whatever the retention time, richness indexes are similar.

As a conclusion, we have seen that changing retention time affects the composition of the cultured ecosystem and that some retention time may be favored to select specific microbiota profiles. Results for mixes show mostly that no differences are observed between fermenters proportions. For instance, mixing fermenters allowed a normalization of the results and each index gets closer to baseline values when fermenters are mixed.

### Example 4: Investigation of Re-Culture Impact on Microbiota Fermentation

Scope of the Experiment

The purpose of the experiment was to investigate the effect of several successive re-cultures on microbiota fermentation.

Protocol

The protocol was identical to example 1 at the exception that flow cytometry analysis differed.
Flow cytometry analysis

For flow cytometry analysis, samples were labelled with a live/dead kit and analyzed on a Guava® easyCyte™ 5HT flow cytometer.
Run performed

Four runs were performed successively:
Run 1: 1^{st} culture
Run 2: 1^{st} re-culture
Run 3: 2^{nd} re-culture
Run 4: 3^{rd} re-culture

In this example, for each run, three fermentations were carried out in parallel in three different fermenters. The difference between these three fermenters was the pH set point, which was set to 5.3, 6.3 and 7.3, respectively. Runs were performed successively. Inoculum of Run 1 was a human fecal microbiota (as described in example 1) and inoculum of following runs was the obtained mix harvested from the three fermenters (mix in 34%/33%/33% proportions) of the previous run. In this way, four cultures were performed in total.

Results

Only mixes (34%/33%/33%) obtained at the end of each run will be presented
Taxonomic composition at the phylum level

Results are shown on FIG. 22

As shown on FIG. 22, whatever the number of cultures, mixes obtained are mainly composed of Firmicutes and Bacteroidetes phyla with less than 3% of Proteobacteria and Fusobacteria.
Butycore

Butycore represents the abundance of bacteria known to produce butyrate. Results are shown in FIG. 23.

As shown on FIG. 23, the butycore index, whatever the number of cultures, is similar and values are like or higher the baseline one.
Core Microbiota

Core microbiota represents the total abundance of following genera: Ruminococcus, Faecalibacterium, Dorea, Corprococcus, Blautia, Alistipes, Bacteroides, Subdoligranulum, Roseburia, Parabacteroides and Lachnospira. Results are shown on FIG. 24.

As shown on FIG. 24, some slight variations are observed in core microbiota abundance depending on re-culture number. Obtained values, whatever the number of cultures, are higher than the baseline one.

Discussion and Conclusion

Results show that successive re-cultures did not affect the metagenomic profiles obtained.

As a conclusion, we have seen that re-cultures did not drastically affect the composition of the cultured ecosystem.

What is claimed is:

1. A method of expanding a complex community of microorganisms comprising the following steps:
(a) cultivating said complex community of microorganisms in at least two bioreactors, preferably in at least three bioreactors, wherein said bioreactors have at least one different parameter, said parameter being selected from pH, temperature, pressure, cultivation time, retention time, gassing conditions, redox potential, and combination thereof, to obtain at least two harvested inocula, and
(b) mixing the at least two harvested inocula obtained in step (a) to obtain an expanded complex community of microorganisms.

2. The method of claim 1, characterized in that said bioreactors have different retention time comprised between 6 hours and 102 hours, preferably between 12 hours and 96 hours, preferably between 24 hours and 72 hours.

3. The method of claim 1, characterized in that at least one bioreactor has a retention time comprised between 6 hours and 18 hours, preferably between 9 hours and 15 hours and more preferably between 11 hours and 13 hours.

4. The method according to claim 1, characterized in that at least one bioreactor has a retention time comprised between 18 hours and 30 hours, preferably between 21 hours and 27 hours and more preferably between 23 hours and 25 hours.

5. The method according to anyone of claims claim 1, characterized in that:
a first bioreactor has a retention time comprised between 18 hours and 30 hours, preferably between 21 hours and 27 hours and more preferably between 23 hours and 25 hours,
a second bioreactor has a retention time comprised between 6 hours and 18 hours, preferably between 9 hours and 15 hours and more preferably between 11 hours and 13 hours, or a retention time comprised between 42 hours and 54 hours, preferably between 45 hours and 51 hours and more preferably between 47 hours and 49 hours.

6. The method according to claim 1, characterized in that:
a first bioreactor has a retention time comprised between 6 hours and 18 hours, preferably between 9 hours and 15 hours and more preferably between 11 hours and 13 hours,
a second bioreactor has a retention time comprised between 18 hours and 30 hours, preferably between 21 hours and 27 hours and more preferably between 23 hours and 25 hours, and
a third bioreactor has a retention time comprised between 42 hours and 54 hours, preferably between 45 hours and 51 hours and more preferably between 47 hours and 49 hours.

7. The method according to claim 1, characterized in that said bioreactors have different pH set points comprised between 4.5 and 8.0, preferably between 5 and 7.6 and more preferably between 5.2 and 7.4.

8. The method according to claim 1, characterized in that at least one bioreactor has a pH set point comprised between 4.5 and 5.8, preferably between 5 and 5.6 and more preferably between 5.2 and 5.4.

9. The method according to claim 1, characterized in that:
a first bioreactor has a pH set point comprised between 4.5 and 5.8, preferably between 5 and 5.6 and more preferably between 5.2 and 5.4,
a second bioreactor has a pH set point comprised between 5.9 and 6.8, preferably between 6 and 6.6 and more preferably between 6.2 and 6.4, or a pH set point comprised between 6.9 and 8, preferably between 7 and 7.6 and more preferably between 7.2 and 7.4.

10. The method according to anyone of claim 1, characterized in that:
a first bioreactor has a pH set point comprised between 4.5 and 5.8, preferably between 5 and 5.6 and more preferably between 5.2 and 5.4,
a second bioreactor has a pH set point comprised between 5.9 and 6.8, preferably between 6 and 6.6 and more preferably between 6.2 and 6.4, and
a third bioreactor has a pH set point comprised between 6.9 and 8, preferably between 7 and 7.6 and more preferably between 7.2 and 7.4.

11. The method according to claim 1, characterized in that the complex community of microorganisms used in step (a) is under the form of a suspension comprising an aqueous saline solution, at least one cryoprotectant and/or at least one bulking agent.

12. The method according to claim 1, characterized in that step (a) or steps (a) and (b) are repeated at least once, with the same pH or with different pH as those previously used in step (a).

13. The method according to claim 1, characterized in that at least one part or all of the complex community of microorganisms used in step (a), at least one part or all of the harvested inocula obtained in step (a) and/or at least one part or all the expanded complex community of microorganisms obtained in step (b) has been frozen, thawed and/or lyophilized.

14. The method according to claim 1, characterized in that the complex community of microorganisms is a faecal microbiota coming from at least one donor, preferably coming from at least two donors.

15. An expanded complex community of microorganisms obtained by the method according to claim 1, characterized in that it comprises:
at least three bacterial phyla selected from Bacteroidetes, Synergistetes, Firmicutes, Proteobacteria, Actinobacteria, Verrucomicrobia, Tenericutes, Fusobacteria and mixtures thereof, or
at least 15 genera selected from the group comprising Clostridium, Eubacterium, Anaerostipes, Bacteroides, Bifidobacterium, Blautia, Butyricicoccus, Collinsella, Dialister, Dorea, Eisenbergiella, Escherichia-Shigella, Faecalibacterium, Fusicatenibacter, Hungatella, Lachnoclostridium, Parasutterella, Roseburia, Eubacterium, [Eubacterium] coprostanoligenes group, [Eubacterium] fissicatena group, [Ruminococcus] torques group, Allisonella, Bilophila, Christensenella, Christensenellaceae R-7 group, Clostridium, Clostridium sensu stricto 1, Coporococcus, Desulfovibrio, Enterococcus, Flavonifractor, Lachnospiraceae NK4A136 group, Lachnospiraceae UCG-001, Lactobacillus, Parabacteroides, Peptostreptococcus, Romboutsia, Ruminoclostridium, Ruminococcaceae UCG-003, Ruminococcaceae UCG-013, Ruminococcaceae UCG-014, Ruminococcus, Subdoligranulum and Sutterella and mixtures thereof.

16. (canceled)

17. A pharmaceutical formulation comprising the expanded complex community of microorganisms obtained by the method according to claim 1.

18. An expanded complex community of microorganisms obtained by:
(a) cultivating said complex community of microorganisms in at least two bioreactors, preferably in at least three bioreactors, wherein said bioreactors have at least one different parameter, said parameter being selected from pH, temperature, pressure, cultivation time, retention time, gassing conditions, redox potential, and combination thereof, to obtain at least two harvested inocula, and
(b) mixing the at least two harvested inocula obtained in step (a) to obtain an expanded complex community of microorganisms;
where, the expanded complex community of microorganisms comprise:
at least three bacterial phyla selected from Bacteroidetes, Synergistetes, Firmicutes, Proteobacteria, Actinobacteria, Verrucomicrobia, Tenericutes, Fusobacteria and mixtures thereof,
at least 15 genera selected from the group comprising Clostridium, Eubacterium, Anaerostipes, Bacteroides, Bifidobacterium, Blautia, Butyricicoccus, Collinsella, Dialister, Dorea, Eisenbergiella, Escherichia-Shigella, Faecalibacterium, Fusicatenibacter, Hungatella, Lachnoclostridium, Parasutterella, Roseburia, Eubacterium, [Eubacterium] coprostanoligenes group, [Eubacterium]fissicatena group, [Ruminococcus] torques group, Allisonella, Bilophila, Christensenella, Christensenellaceae R-7 group, Clostridium, Clostridium sensu stricto 1, Coporococcus, Desulfovibrio, Enterococcus, Flavonifractor, Lachnospiraceae NK4A136 group, Lachnospiraceae UCG-001, Lactobacillus, Parabacteroides, Peptostreptococcus, Romboutsia, Ruminoclostridium, Ruminococcaceae UCG-003, Ruminococcaceae UCG-013, Ruminococcaceae UCG-014, Ruminococcus, Subdoligranulum and Sutterella and mixtures thereof, or
a pharmaceutical formulation comprising expanded complex community of microorganisms; and
where the expanded complex community of microorganisms is employed for its use:
as a medicament optionally in combination with drug-based therapy, immunotherapy, immune checkpoint inhibitors, therapy targeting tumor neoantigens, chemotherapy and/or radiation therapy,
in the prevention and/or treatment of septicaemia, septic shock, infection such as Clostridioides difficile infection and associated diarrhoea (CDI), ulcerative colitis, inflammatory bowel disease (IBD), irritable bowel syndrome (IBS), idiopathic constipation, Coeliac disease, Crohn's disease, type I diabetes, type II diabetes, food allergies, solid and liquid cancers, Graft-versus-host disease (GvHD) such as gastrointestinal acute GvHD, steroid-resistant GvHD or steroid-dependant GvHD, obesity and morbid obesity, autism, sclerosis, chronic vaginal infection such as cystitis and mycosis, bone and joint infections, Parkinson's disease, Alzheimer's disease, schizophrenia and bipolar disorders, gastrointestinal disorders such as intestinal inflammation, diarrhoea, traveller's diarrhoea, mucositis, abdominal pain and gastrointestinal bleeding, intestinal dysbiosis such as intestinal dysbiosis associated with cancer chemotherapy or immunotherapy, intensive care unit (ICU) related dysbiosis, alcoholic and non-alcoholic liver disease, treatment-induced inflammation such as treatment-induced gut inflammation, inflammation induced by anti-cancer therapy, hematologic disease, viral coronavirus infections, infectious or non-infectious complications resulting from an allogenic hematopoietic stem cell transplantation (allo-HSCT) such as steroid-refractory graft versus host disease (SR-aGvHD) following allogenic hematopoietic stem cell transplantation (allo-HSCT), carrying or infection by multi-resistant bacteria including Clostridium, Enterococci such as vancomycin-resistant enterococci (VRE) and Glycopeptide-resistant Enterococci (GRE), bacteria producing extended-spectrum beta-lactamases (ESBL), carbapenemase-producing Enterobacteriaceae (CPE), Emerging Bacteria Highly Resistant to Antibiotics (BHRe), and/or malignant hemopathy, and combination thereof, optionally in combination with drug-based therapy, immunotherapy, immune checkpoint inhibitors, therapy targeting tumor neoantigens, chemotherapy and/or radiation therapy, or
in microbiome ecosystem therapy, optionally in combination with drug-based therapy, immunotherapy, immune checkpoint inhibitors, therapy targeting tumor neoantigens, chemotherapy and/or radiation therapy.

19. (canceled)

20. (canceled)
